SEQUENCE LISTING (37 sequences)

SEQ ID NO: 1 - DNA, 4826 bp, Triticum aestivum
  source 1..4826: mol_type = genomic DNA
atgtggaagctcaagattgctgagggtggcccgtggctcacgagcggcaacaatcacgtcggaagagaaacatgggagtttgaccaaaatgacaccggatcaagcgaagagcgggatgcggtcgacgcttcacgggccgaattccagaagaacaggttcaggacaaggcacagctccgatgttttggcccgcatgcaggtaattaaaatgcaccgatatatacacaccttgtcttggtggtatctatccaactgactatttgaagcgtttttatgaaactgagtacatgctctgctttttgttgtgcagctagcaaaggggaataagttcagccttgaccaacaacagaaaccaaaagatgatgaaactagtggtgatatcaatatagctacggtgtcggagacactgagaagggcactcagatacttctcagccgtacaagcgcatgatgggcactggccaggagattttccggggcctctctttaccacagcaaccatggtaattaagatgcatcatacataaatttgatgtttgtttgtttgcatcatcatcaccatcaagttagttatgataaacatttatttagatggttatgcgtgctatgtattcttgctaactattcacaactttggtgtgacatcgacatgggagacattctcatgtttgttcattcctgcttgctgcagatcatagttttgtatgtcacggagtcgttaagtactacgctatcgtcagaacaccgcaaggagatctgtcgctatctgtacaaccgacaggtaaggtgcacgccacatttgccaggaattaatcttgggtaacttttagcaagttctgttttccacaatatatatttttcactaaaactgtttctacggatgtcttcaaaaaatttacacaagaaaattatatcggaataaatggactgacccaagtttttgtattatcagaatatggatggaggatggggactacatgcggaaggtgagagctccatgctcagctcggctctcaactataccgctctaagactacttggtgagggtgctgatgatggaccagatatgtccatgccaaaagcaaggaaatggatacatgaccatggtggtacaacaatgataccaatcttgggaaaagtctggctctcggtaacaatcatcctccttgtgccagccccaagtttctattttcagcaagtgattcaccagttagcttgatttgccaaacatttcaccacttcttgttaatatactcagagtaaaaattatgttcttgttaacaagcaagtctgaaactgcatgcttccagttatgcagaattcttcaacaagagtaaaagtttgacaaaactgtttatgtcgatcgacccaaagcgcatctgaactatatacctgatgctgcaggtacttggggtttttgaatggtcaggtgtaaaccctatgcccccagaattgttccttctgccatccttcgttcctattcaaccaggtacgcagagttcttcgccggcgcgttaagtgttcaaccatgttacgacaattaattacttggttattcaacactaggtattaaccatattaggaagaacttattttatgagaacaatcatgtcgtttctcaaatgcaggacggttgtggagtcacttcagaatggctttcatccccatgtcctatttatatggcaagaagtttgttggcccaacaacaaaactggttttatcattaagagaagagctgcatattcacccctataaaaagattaattggaagcaagcacgaaaattatgcgcaaaggtgagtccactaactatatagttttaccttacagttaaattttgaaggattggtgacagcagattatgaattcaaatgcaggaagatgcctatcatccacatacctggctccaagaatgcttgtccgattgcctttacagtttcggtgaatcttttctggcatgttggccagtttcctacatgagacgaaaagctctaggacaaattgccgatttcctgaaatacgaagatgatatttcacggtatatctgcatcggcgccgcacaaaaggttagacttacggaggtcttcacaaaataattcagtgttgattttcagtgagctgtaaatgaacatcatttccttacaaagaataaacattattacaggcattatccatgttatgttgttggtctgagaatcccaattcagatgcattcaagcaccacttggctagagttgctgatttcctatggctcggcgaagatggcatgaaagtgcgggtaaattttaataactcttgtttctataatattcagggtgaagatacctttccatttaaaatgcatctcagtcaaattgaaatttgtgcgcaaactaactgatattacatgacttcattgttcattcaaaaggtatgtgcgggtcaatcttgggatgttgctttcgccgtacaagcgatattggcatgtgatgttgcaggggaatttggaagtactctcaagaaagcacatcatttcctaaaagcatcccaggtattgtatttccaaaactagattttgttacttatatttatcaggacttaaatcctaacactactaacttcattttttttagattgtggacaacccttctggtgacttcagcagaaagtaccgccatatctctaaaggaggatgggccttccaggttgcagatcagggttggcaggtttcagattgcacagcagaagctcttaaggtgcgatatcaaattaactaaagatgcaaaaagagtgaacagaactaaagtggaaacatgcctttcattttgctactacaaagtaatgcatttcattttgtttcagcccttcaggcatttgtaattcttcttctgtttcaggctctgttactgctctcaaagtttccgtcagatatcgtgggtgatcagatggaaacatgccgcttccatgatgcagtgaacgtattactatctttacaggtgtgtaaccatatgtaaagaagcatattagcaactattgccacagtagagttgggtaaatgtagttttcagtttgacgctaaagcactaattcagcaccacagtaaatggtaatgaaaggcagattcagtgtgagcactgtcaactcttacgtgcatgattttgatatgcaatctttgcaaacaatttggccattttgaatagtgtgctatactgaaaccaagagcccatgctttcttgctaaaaaaatgacgtagaattaacccatagcattttggctcatgcaacagatattgcagtcctgttacagatttacaccaatgccaagcacatcacaggttctgtaaaaatgatttgacactaggttggatgcagaatcctaatggtggctatggaacttgggagctagctcgtacatatccatggatggaggcaagtgtcttgtaaaacacctgcacttcatgctattattatttggagtttaggaaacccaatttaacattgggtaatgagacttttaatatattttttccttttttcagaatttaaacatgacagagatatatgcagacatcatggtggagcatcagtaagaaatctacaacccttcttagcaatagctatcacatgtgactcgatattaagacacatacatattgcaggtacgtcgagtgtacctcgtcggtcatccaagcattggccctgtttcggcaaaaatacccaagccatcgggaagatgaaatagaacaatgcatcaggagagcgacagagttcatcgagaagttgcagaatgaggacggttcatggtcagtgtactgtcgacaattttatagtgttttatattaacaaggagaaggtgtgttgttgacacattgatcaataagttgttttgcaggttcggatcatggggtatttgcttcacatacggcacgtggtttgctatagagggcctatcagcagttggacagtgttacaataatagcacctacatccggaagggttgccagtttctattatcgaagcagctaaggaatggtggatggggtgagagtcatctttcatccacaaccaaggtaaatgctctggaatgacttattatgttgatggaaagcaagcatcctaaacaatgagaaaatcaagcatcctcacaaagaaaaggatgtctttattcttgcttgaatataccttgaaacaagatactggcaaaacattctgggctttctacggtaactgaccacagatgtcatctcaaaatctaaacaggcatacacaaacctagacggggagaaatcacatatagtcaacacggcatgggcaatgttggcactaatgaaagctggacaggtgaccacttctctttctctctccaaaggtttcacgtggtcaaattccaaaagaccagtattatgttaatgcgcagtatggtaaatgcacctttggccccagaatagttgaaccgatctatagtataacttctaattcagttgaactacgtagtactatcgaattaattccagcttttcttcgacttcaggccgaaagagatccgtctcctttgcacgaagctgcaaggcttatcatgagcatgcagcttggcaatggtgacttcccacaggaggtaactgctacaattaatgtaccatgtgttacacgatttaaaccaaaatcttgaattcttgtgttcttaatactataataaaaatgattggggataactcaattttgtatattattatgtaggaaatgattggaagtttcttgaaaaatggtcccttgtgttatatggcttatcgcaacatattccccatatgggcccttggagagtatcatagattagtactttgctctggctaa

SEQ ID NO: 2 - DNA, 2280 bp, Triticum aestivum
  source 1..2280: mol_type = other DNA
atgtggaagctcaagattgctgagggtggcccgtggctcacgagcggcaacaatcacgtcggaagagaaacatgggagtttgaccaaaatgacaccggatcaagcgaagagcgggatgcggtcgacgcttcacgggccgaattccagaagaacaggttcaggacaaggcacagctccgatgttttggcccgcatgcagctagcaaaggggaataagttcagccttgaccaacaacagaaaccaaaagatgatgaaactagtggtgatatcaatatagctacggtgtcggagacactgagaagggcactcagatacttctcagccgtacaagcgcatgatgggcactggccaggagattttccggggcctctctttaccacagcaaccatgatcatagttttgtatgtcacggagtcgttaagtactacgctatcgtcagaacaccgcaaggagatctgtcgctatctgtacaaccgacagaatatggatggaggatggggactacatgcggaaggtgagagctccatgctcagctcggctctcaactataccgctctaagactacttggtgagggtgctgatgatggaccagatatgtccatgccaaaagcaaggaaatggatacatgaccatggtggtacaacaatgataccaatcttgggaaaagtctggctctcggtacttggggtttttgaatggtcaggtgtaaaccctatgcccccagaattgttccttctgccatccttcgttcctattcaaccaggacggttgtggagtcacttcagaatggctttcatccccatgtcctatttatatggcaagaagtttgttggcccaacaacaaaactggttttatcattaagagaagagctgcatattcacccctataaaaagattaattggaagcaagcacgaaaattatgcgcaaaggaagatgcctatcatccacatacctggctccaagaatgcttgtccgattgcctttacagtttcggtgaatcttttctggcatgttggccagtttcctacatgagacgaaaagctctaggacaaattgccgatttcctgaaatacgaagatgatatttcacggtatatctgcatcggcgccgcacaaaaggcattatccatgttatgttgttggtctgagaatcccaattcagatgcattcaagcaccacttggctagagttgctgatttcctatggctcggcgaagatggcatgaaagtgcgggtatgtgcgggtcaatcttgggatgttgctttcgccgtacaagcgatattggcatgtgatgttgcaggggaatttggaagtactctcaagaaagcacatcatttcctaaaagcatcccagattgtggacaacccttctggtgacttcagcagaaagtaccgccatatctctaaaggaggatgggccttccaggttgcagatcagggttggcaggtttcagattgcacagcagaagctcttaaggctctgttactgctctcaaagtttccgtcagatatcgtgggtgatcagatggaaacatgccgcttccatgatgcagtgaacgtattactatctttacagaatcctaatggtggctatggaacttgggagctagctcgtacatatccatggatggagaatttaaacatgacagagatatatgcagacatcatggtggagcatcagtacgtcgagtgtacctcgtcggtcatccaagcattggccctgtttcggcaaaaatacccaagccatcgggaagatgaaatagaacaatgcatcaggagagcgacagagttcatcgagaagttgcagaatgaggacggttcatggttcggatcatggggtatttgcttcacatacggcacgtggtttgctatagagggcctatcagcagttggacagtgttacaataatagcacctacatccggaagggttgccagtttctattatcgaagcagctaaggaatggtggatggggtgagagtcatctttcatccacaaccaaggcatacacaaacctagacggggagaaatcacatatagtcaacacggcatgggcaatgttggcactaatgaaagctggacaggccgaaagagatccgtctcctttgcacgaagctgcaaggcttatcatgagcatgcagcttggcaatggtgacttcccacaggaggaaatgattggaagtttcttgaaaaatggtcccttgtgttatatggcttatcgcaacatattccccatatgggcccttggagagtatcatagattagtactttgctctggctaa

SEQ ID NO: 3 - DNA, 4956 bp, Triticum aestivum
  source 1..4956: mol_type = genomic DNA
atgtggaagctcaagatcgccgagggcggcccgtggctctcgagcggcaacaatcacgtcggaagagaaacatgggagtttgaccaaaacgatgccggatcaagcgaagagcgggatgcggtcgacgctgcacgggccgaattccagaagaacaggttcaggacaaggcacagctccgatgttttggctcgcatgcaggtaactaaaatgcactgatatgtatgcacctttgcggtctctgatgatatttgaagctttttattataagaaactgagtacatgctctgtttttcgttctgcagctagcaaagaagaataacttcagccttgatcagcagaaaccaaatgatgaagctactgtcgacatcaatgtagctacggtatcagaggcactgagaagggcactcggatacttctccgccatacaagcgcatgatgggcactggccaggagattttccggggccactctttaccacagcaaccatggtaattaagatacatcataattaaaatttctgtttaattgtttgggtcatcaccatcaagttagttatagccaacatttagatgctcatgcacactgtattctcatattcttgcacttccacatgacattcttatgcttattccttcctgctgcagatcatagttttgtatgtcaccgagtcattaggtactacgctgtcatcggagcatcgcaaggagatctgtcgttacttgtacaaccgtcaggtaattaagctgcacattttccaggaaactagtcatggataactaggaagttctgttttccacaatatatattttgactgaaactgtttctctggatgtcttcaaaaaattttagaagaaaaatatatcatcaaaaatagactgacccaatttttgtatcatcagaatatagatggaggctggggactacacgcagaaggcgaaagctccatgctcagctcagctctcaactacactgctctaagactacttggtgagggcgctgatgatggaccggatatgtccatgccgaaagctcggaaatggatacatgaccatggtggtgcaacaatgataccaatcttgggaaaagtgtggctctcggtaacaatcatcacccttgtgccggccccaactttttattttcagcaagtgattcacaagttagcttgattcgccaaacatttcaccgcttcttgttaatatactcggagtaaaaattatgttcttgttaacaagcaagtctaaaactgcatgctttcagttatgcagaattcttcaacaagagtaaaaatttgacgaaactatttatgtcgatcgacccaaagcgcatctgaactgtgtgcatgcctgatgctgcaggtacttggggtttttgaatggtcaggtgtaaaccctatgcccccagaattgttccttctgccatccttcgttcctattcaaccaggtacgcagagttcttcgccggcgcgttaagtgttcaaccatgtcacgacagttaattacttggtaggtctgaaccacattaggaagagcttattttatgagaacaatcatgttgtttctcaagtgcaggacggttgtggagtcacttcagaatggctttcatccccatgtcctatttatatggcaagaagtttgttggcccaataacaaaactggttttgtcattaagagaagagctgcatattcacccctacaaaaagattaactggaagcaagcgcgcaaattatgcgcaaaggtgagtccactaaactatatagttttaccgcttgttcggcaaatattccacgtgattttacacttagatttaatttcgtatgtagagttctgatctgtcacaattaccatttgtcctcgcaactagaatcaactgtcagtgaatttcttgttcaattttaaaggatcggtgacaacagattatgaattcaaatgcaggaagatgcctatcatccacatacctggctccaagaatgcttgtctgactgcctttacagtttcggtgaaccgtttctggcatgttggccggtttcctacatgagacgaagagctctacgacaaattgccgatttcctgaaatacgaagatgatatttcacggtatatctgcatcggcgccgcgcaaaaggtaaggcttacggaggtcttcacaaaataattcagtgctgggttttcagtgagctgtaaatgaacatcatttcctgacagagaataaacattattacaggcattatccatgttatgctgttggtctgaggatcccaattcagatgcattcaagcgccacttggctagagtcgctgatttcctatggctcagtgaagatggcatgaaagtgcgggtaaattttaataactcttgtttatgtaatatagtccatttatgtatcccagtcaaactgaaatttgtgcacaaactaactgatattacacgacttcattgttcgttcgaaaggtatgtgcgggccaatcatgggatgttgcttttgccgtacaagcgatattagcgtctaatgttgcagaggaatttggaactactctcaggaaagcacatcatttcataaaagcatcccaggtaatatatttttaccagaactgaatcttgttacttatatttatcttgacttaatccctaatcctactaccttaattttgttaagattgtggacaatccttcaggtgacttcgccagaaagtaccgtcacatctctaaaggaggatgggccttccaggttgcagaccagggctggcaggtttcagattgcacagcagaagctctcaaggtgcgataccaaattaactaaagatgcaaaagaaagtgaacaaaactaaaatggaaacatgcctttcattttgctactacaaagtaatgcatttcattttgtttcagcctttcaggcttttgtaattcttcttctgtttcaggctctgttactgctctcgaagtttccgtcagatatcgtgggtgatcagatggaaacatgccgcttccatgatgcggtgaacgtattactatctttacaggtgtgtaaccacatgttgtacttcagcagttccgctacaaatgtacaccaatgccaagcaccgcgcagagtaaaaatgatttgacacttaggttgtatgcagaatcctaatggtggctatggaacttgggagctagctcgtacatatccatggatggaggcaagtgttctggaaacacttgcgctgtgcgctatttttatttggaatttaggcatctcaatttaacatgggtaatgagatcccgcaaaaaaaaacatgggtaatgagacttctaacgttttccccttttcagaatttaaacatgacagagatatatgcggacatcatggtggagcatcagtaagatatgtacaacctttcttagcaatagctcttgcatgtgactcgatattaagatgcacacatatcgcaggtacgtcgagtgtacctcatcggtcatccaagcattggccctgtttcggcaaaagtaccccgggcatcgggaagatgaaatagaacagtgcatcaggagagcgacagaattcattgagaagttgcagaatgaagacggttcatggtcagtatactatcaacagtgttatagtgtttatattaacatggagaaggtgacacattgctgctgcttgggttgccctttaagttttcacattacactgaccaaatattgataattatagctgatgaaactagaaagaaattaattaaacaaatggcacatattgaacttttggcaaaatgttgcaacttctcttgatggagaaattgataatcaatgatcaataagttgttttgcaggtttggatcatggggtatttgcttcacatatggcacatggtttgctatagagggcctatcagcagttggacagtgttacaataacagcacctacattcggaagggttgccagtttctattatcaaagcagctaaggaatggtggatggggtgagagccatctttcatccacaaccaaggtaaacgctgtcgaatggcttcttatgttgatgaacagaaagtgctcacttctttgagaaaatcaagcatcctgacaaagaaaatattctttattcttctttttatctttttttgaggggttattctttcttacccgcaaaaaaaaaaagagaggggttattctttcttcatttttttcttcaatataagttgaaacaagatactggcaaaaaagtctgcactttctacggtaactgaccacagatgtcatctcaaaatctaaacaggcatacaccaacctagacggggagaaatcacacatagtcaacaccgcatgggcgatgttggcgcttatgaaagctggacaggtgatcacttctctttctctctccaaaggtttcacacggtcaaattccaaaagactagtattatggtaaagcgcagtatggttaatgcacctttggccccagaatagttgaaccgatctatagtataacttctgatcttggaaaaaattataggagaacttcaaggtgcaggttctaattcagttgaactacgaagtagtatcgaattaatcccagcttttcttcgacttcaggccgaaagagatccgtctcctttgcacgaagctgcaagacttatcatgagcatgcagcttggcaatggtgacttcccacaggaggtaactgctacaattaatgtttttttctttcaaaaatgaggattacccccggcctctccatcgagtttgctacaattaatgtaccatggcgttacacgatttaaaccgaaatcttgaattcttgttttcttaataattctatagtaaaaatgattggagctgactcaatcttgtatattatcatgtaggaaatgattggaagtttcttgaaaaatggtcccttgtgttatatggcttatcgcaacatattccccatatgggcccttggagagtatcatagattagtactttgctctggctaa

SEQ ID NO: 4 - DNA, 2274 bp, Triticum aestivum
  source 1..2274: mol_type = other DNA
atgtggaagctcaagatcgccgagggcggcccgtggctctcgagcggcaacaatcacgtcggaagagaaacatgggagtttgaccaaaacgatgccggatcaagcgaagagcgggatgcggtcgacgctgcacgggccgaattccagaagaacaggttcaggacaaggcacagctccgatgttttggctcgcatgcagctagcaaagaagaataacttcagccttgatcagcagaaaccaaatgatgaagctactgtcgacatcaatgtagctacggtatcagaggcactgagaagggcactcggatacttctccgccatacaagcgcatgatgggcactggccaggagattttccggggccactctttaccacagcaaccatgatcatagttttgtatgtcaccgagtcattaggtactacgctgtcatcggagcatcgcaaggagatctgtcgttacttgtacaaccgtcagaatatagatggaggctggggactacacgcagaaggcgaaagctccatgctcagctcagctctcaactacactgctctaagactacttggtgagggcgctgatgatggaccggatatgtccatgccgaaagctcggaaatggatacatgaccatggtggtgcaacaatgataccaatcttgggaaaagtgtggctctcggtacttggggtttttgaatggtcaggtgtaaaccctatgcccccagaattgttccttctgccatccttcgttcctattcaaccaggacggttgtggagtcacttcagaatggctttcatccccatgtcctatttatatggcaagaagtttgttggcccaataacaaaactggttttgtcattaagagaagagctgcatattcacccctacaaaaagattaactggaagcaagcgcgcaaattatgcgcaaaggaagatgcctatcatccacatacctggctccaagaatgcttgtctgactgcctttacagtttcggtgaaccgtttctggcatgttggccggtttcctacatgagacgaagagctctacgacaaattgccgatttcctgaaatacgaagatgatatttcacggtatatctgcatcggcgccgcgcaaaaggcattatccatgttatgctgttggtctgaggatcccaattcagatgcattcaagcgccacttggctagagtcgctgatttcctatggctcagtgaagatggcatgaaagtgcgggtatgtgcgggccaatcatgggatgttgcttttgccgtacaagcgatattagcgtctaatgttgcagaggaatttggaactactctcaggaaagcacatcatttcataaaagcatcccagattgtggacaatccttcaggtgacttcgccagaaagtaccgtcacatctctaaaggaggatgggccttccaggttgcagaccagggctggcaggtttcagattgcacagcagaagctctcaaggctctgttactgctctcgaagtttccgtcagatatcgtgggtgatcagatggaaacatgccgcttccatgatgcggtgaacgtattactatctttacagaatcctaatggtggctatggaacttgggagctagctcgtacatatccatggatggagaatttaaacatgacagagatatatgcggacatcatggtggagcatcagtacgtcgagtgtacctcatcggtcatccaagcattggccctgtttcggcaaaagtaccccgggcatcgggaagatgaaatagaacagtgcatcaggagagcgacagaattcattgagaagttgcagaatgaagacggttcatggtttggatcatggggtatttgcttcacatatggcacatggtttgctatagagggcctatcagcagttggacagtgttacaataacagcacctacattcggaagggttgccagtttctattatcaaagcagctaaggaatggtggatggggtgagagccatctttcatccacaaccaaggcatacaccaacctagacggggagaaatcacacatagtcaacaccgcatgggcgatgttggcgcttatgaaagctggacaggccgaaagagatccgtctcctttgcacgaagctgcaagacttatcatgagcatgcagcttggcaatggtgacttcccacaggaggaaatgattggaagtttcttgaaaaatggtcccttgtgttatatggcttatcgcaacatattccccatatgggcccttggagagtatcatagattagtactttgctctggctaa

SEQ ID NO: 5 - DNA, 4809 bp, Triticum aestivum
  source 1..4809: mol_type = genomic DNA
atgtggaagctcaagatcgccgagggcggcccgtggctcacgagcggcaacaaccacttcggaagagaaacatgggagtttgaccgaaatgacgctggatcaagcgaagagcgggatgcggtcgacgctgcacgggctgaattccagaagaacaggttcaggacaaggcacagctccgatgttttggcccgcatgcaggtaagtaaaatgcactgatatatacacccttgtcttggtggtatctatccaactgaatatttgaagtgttttttgttgtgcagttagtaaaggggaataacttcagccttgaccaacaacagaaaccaaaaggtgatgaaactagtgtcgacatcaacatagctacggtgtcggagacactgaaaagggcactcagatacttctcagccatacaagcgcacgatgggcactggccaggagattttccgggccctctctttaccacagcaaccatggtaattaagatgcatcatacataaacttgatgtttgtttgtttgcttcatcatgaccatcaagttagttatgataaacatttatttagatggttatgcatgctatatattcttgctaactattcacaactttggcgtgacatcgacatcgacatgggagacattctgatgtttgttcattcctgcttgctgcagatcgtagttttgtatgtcacggagtcgttaggtactacgctatcgtcagaacaccgcaaggagatctgtcgctatttgtacaaccgacaggtaaggtgcacgcacatttgccaggaaattaattatgggtaacttttagcaagttctgttttccacaatatatattttcactaaaactgtttctacggatgtcttcaaaatttttacacaagaaaattatatcggaataaatagactgacccaagtttttgtattatcagaatatggatggaggatggggactacatgcggaaggcgagagctccatgctcagctcagctctcaactacgctgctctaagactacttggtgagggtgctgatgatggaccagatatgtccatgccaaaagctaggaaatggatacatgaccatggtggtgcaacaatgataccaatattgggaaaagtgtggctttcggtaacaatcatcctccttgtgccggccccaagtttttattttcagcaatgattcacaagttagcttgattcgccaaacatttcaccacttcttgttaatatactcaaagagtaaaaattatgttcttgttaacaagcaagtctgaaactgcatgctttcagttatgcagaattcttcaacaagagtaaaaatttgacgaaactatttacgtcgatcgacccaaagtgcatctgaactgtatgcctgacgctgcaggtacttggggtttctgaatggtcaggtgtaaaccctatgcccccagaattgttccttctgccatccttcgttcctatccaaccaggtacgcagagttcttcaccggtgcgttaagtgttcaaccatgttactacaattaattacctggttattcaacactaggtatgaaccatattaggaagagcttattttatgagaacaatcatattgtttctcaaatgcaggacggttgtggagtcacttcagaatggctttcatccccatgtcctatttatatggcaagaagtttgttggcccaataacaaaactggttttatcattaagggaagagctgcatattcacccctacaaaaagattaactggaagcaagcgcgcaaattatgcgcaaaggtgagtccactaactttatagtttttaccttttgttctgcgagtattccacgtgatttcacatttggattaatttcttgttcaattttaaaggattatgaattcaaatgcaggaagatgcgtatcatccacatacctggctccaagaatgcttgtccgattgcctttacagtttcggtgaaccttttctggcatgttggccagtttcccacatgagacgaaaagctctacgacaaattgccgatttcctgaaatacgaagatgatatttcacggtatatctgcattggctctgcgcaaaaggttagacttacggaggtcttcacaaaataattcagtgctgattttcagtgagctgtaaatgaacatcatttccttacaaagaataaacattattacaggcattatccatgttatgctgttggtctgagaatcccagttcagatgcattcaagcgccacttggctagagtttctgatttcctatggctcggtgaagatggcatgaaagtgcgggtaagttttaaaaattcttctttctgtagtacagaatatatccttccatttagtcaaaattgaaatttgtgcacaaactgatattacatgtcattgttcattcaaaaggtatgtgcaggtcagtcatgggatgttgcttttgctgtacaagcaatattagcgtgtgatgttgcacaggaatttggaactactctgaagaaagcacaccatttcataaaagcatcccaggtaatatattttttctagaactgaattttgttacgtacatttatcttgactgaaaccctaactctactaacttacaatttttttttttaagattgtcagcaatcctaccggtgacttcagcagaaagtaccgtcacatctctaaaggaggatgggccttccaggttgcagaccagggctggcaggtttcagactgcacagcagaagctctcaaggtgtgatatcaaattaattaaggatgcaaaagaaagtgaacagaactaaaatggaaacatgcctttcattttgctactacaaagtaatgcatttcattttgtttcagcctttgaggcttttgtaactcctctgtttcaggctctgttactgctctcaaagtttccgtcagagatcgtgggtgatcagatggaaacatgccgcttccatgatgcagtgaacatattattatctttacaggtgtggaaccacaacttgtactttagattctatattttggtctatgcatacacaggggcatgtccataaagttgggtaaatgttagttttcagttactccctccgttcggaattcttctagatacatccatttccaagacaagtaattccgaacggagggagtagatgctagtgaataattcagcacaaggccgtatggctggtaacgaaaggcagattcagtgcgatcagtgtctactcttatgtgcatgattctgatatgcaatctttacttgaagcaagtctgtcaggttgtgtaaaaatgatttgacactaggttgtacgcagaatcctaatggtggctatggaacttgggagctagctcgtacatatccatggatggaggcaagtgctctgcaaacacctgcactccatgttgtttttatttggagtttaggaaaatcaatttaacattgggtaatgagacctttaatatatttttttcttttccagaatttaaacatgacagagatatatgcagacatcatggtggagcatcagtaagaaatctacaatctttcttagcaatagctcttgcatgtgactcgatattaagatgcatacatgtcgcaggtacgtcgagtgtacctcgtcggtcatccaagcattggccctgtttcggcaaaaataccccgggcatcgggaagatgaagtagaacaatgcatcaggagagcgacagaattcatcgagaagttacagaatgaggacggttcatggtcagtacactatcaacagttttatagtgtttatattaagtttattaaacaaggagaaggtgtgctgttgacatattgatcaacaagttgttttgcaggttcggatcatggggtatttgcttcacatacggcacgtggtttgctatagagggcctatcggcagttggacagtgttacaataatagcacctacatccggaagggttgccagtttctattatcaaagcagctaaggaatggtggatggggtgagagtcatctttcatccacaaccaaggtaaacgctatcgaatggcttcttatgttgatgaacagaaagtactcacatcttagagaaaatcaagcataccgacaaagaaatttttaattattcttgtttcaatttaccttgaattctgcactttctacggtaaccgatcatacgtgtcatctcgaattctaaacaggcatacacgaacctagacggggagaaatcacatgtagtcaacaccgcatgggcaatgttggcactaatgaaagctggacaggtgaccacttctctttctctctccaaaggtttcacatggtcaaagtccaaaagactactattatgttaatgcgcagtatcgtaaatgcacgaaaggtttccccagaataactgaaccgatctatagtataacttctgatcttggaaaaaattataggagaacttcaaggtgcaggttctaattcaattgaactacgaagtactgtcgaattaattccagcttttcttcgacatcaggccgaacgagatccgtctcctttgcacgaagctgcaagacttatcatgagcatgcaacttggcaatggtgacttcccacaggaggtaactaactgctacagttaatgtaccatgcgctacacgatttaaaccgaaatcttgaattcttgttttcttaataatgctataataaacatgattggagctgactcaatcttgtatattattatgtaggaaatgattggaagtttcttgaaaaatggtcccttgtgttatatggcttatcgcaacatattccccatatgggcccttggagagtatcatagactagtactttgctctggctaa

SEQ ID NO: 6 - DNA, 2280 bp, Triticum aestivum
  source 1..2280: mol_type = other DNA
atgtggaagctcaagatcgccgagggcggcccgtggctcacgagcggcaacaaccacttcggaagagaaacatgggagtttgaccgaaatgacgctggatcaagcgaagagcgggatgcggtcgacgctgcacgggctgaattccagaagaacaggttcaggacaaggcacagctccgatgttttggcccgcatgcagttagtaaaggggaataacttcagccttgaccaacaacagaaaccaaaaggtgatgaaactagtgtcgacatcaacatagctacggtgtcggagacactgaaaagggcactcagatacttctcagccatacaagcgcacgatgggcactggccaggagattttccgggccctctctttaccacagcaaccatgatcgtagttttgtatgtcacggagtcgttaggtactacgctatcgtcagaacaccgcaaggagatctgtcgctatttgtacaaccgacagaatatggatggaggatggggactacatgcggaaggcgagagctccatgctcagctcagctctcaactacgctgctctaagactacttggtgagggtgctgatgatggaccagatatgtccatgccaaaagctaggaaatggatacatgaccatggtggtgcaacaatgataccaatattgggaaaagtgtggctttcggtacttggggtttctgaatggtcaggtgtaaaccctatgcccccagaattgttccttctgccatccttcgttcctatccaaccaggacggttgtggagtcacttcagaatggctttcatccccatgtcctatttatatggcaagaagtttgttggcccaataacaaaactggttttatcattaagggaagagctgcatattcacccctacaaaaagattaactggaagcaagcgcgcaaattatgcgcaaaggaagatgcgtatcatccacatacctggctccaagaatgcttgtccgattgcctttacagtttcggtgaaccttttctggcatgttggccagtttcccacatgagacgaaaagctctacgacaaattgccgatttcctgaaatacgaagatgatatttcacggtatatctgcattggctctgcgcaaaaggcattatccatgttatgctgttggtctgagaatcccagttcagatgcattcaagcgccacttggctagagtttctgatttcctatggctcggtgaagatggcatgaaagtgcgggtatgtgcaggtcagtcatgggatgttgcttttgctgtacaagcaatattagcgtgtgatgttgcacaggaatttggaactactctgaagaaagcacaccatttcataaaagcatcccagattgtcagcaatcctaccggtgacttcagcagaaagtaccgtcacatctctaaaggaggatgggccttccaggttgcagaccagggctggcaggtttcagactgcacagcagaagctctcaaggctctgttactgctctcaaagtttccgtcagagatcgtgggtgatcagatggaaacatgccgcttccatgatgcagtgaacatattattatctttacagaatcctaatggtggctatggaacttgggagctagctcgtacatatccatggatggagaatttaaacatgacagagatatatgcagacatcatggtggagcatcagtacgtcgagtgtacctcgtcggtcatccaagcattggccctgtttcggcaaaaataccccgggcatcgggaagatgaagtagaacaatgcatcaggagagcgacagaattcatcgagaagttacagaatgaggacggttcatggttcggatcatggggtatttgcttcacatacggcacgtggtttgctatagagggcctatcggcagttggacagtgttacaataatagcacctacatccggaagggttgccagtttctattatcaaagcagctaaggaatggtggatggggtgagagtcatctttcatccacaaccaaggcatacacgaacctagacggggagaaatcacatgtagtcaacaccgcatgggcaatgttggcactaatgaaagctggacaggccgaacgagatccgtctcctttgcacgaagctgcaagacttatcatgagcatgcaacttggcaatggtgacttcccacaggaggaaatgattggaagtttcttgaaaaatggtcccttgtgttatatggcttatcgcaacatattccccatatgggcccttggagagtatcatagactagtactttgctctggctaa

SEQ ID NO: 7 - DNA, 4968 bp, Triticum aestivum
  source 1..4968: mol_type = genomic DNA
atgtggaagctcaagattgctgagggtggcccgtggctcacgagcggtaacaatcacgtcggaagagaaacatgggagtttgaccaaaatgacaccggatcaagcgaagagcgggatgcggtcgacgcttcacgggccgaattccagaagaacaggttcaggacaaggcacagctccgatgttttggcccgcatgcaggtaattaaaatgcaccgatatatacacaccttgtcttggtggtatctatccaactgaatatttgaagcgtttttatgaaactgagtacatgctctgctttttgttgtgcagctagcaaaggggaataagttcagccttgaccaacaacagaaaccaaaagatgatgaaactagtggtgatatcaatatagctacggtgtcggagacactgagaagggcactcagatacttctcagccgtacaagcgcatgatgggcactggccaggagattttccggggcctctctttaccacagcaaccatggtaattaagatgcatcatacataaatttgatgtttgtttgtttgtatcatcatcaccatcaagttagttatgataaacatttatttagatggttatgcgtgctatatattcttgctaactattcacaactttggtgtgacatcgacatgggagacattctcatgtttgttcattcctgcttgctgcagatcatagttttgtatgtcacggagtcgttaggtactacgctatcgtcagaacaccgcaaggagatctgtcgctatctgtacaaccgacaggtaaggtgcacgtacattttccaggaaattaatcatgggcaacttttagcaagttctgttttccacaatatatattttcactaaaactgtttctacgtatgtcttcaaaattttacacaagaaaaaattatatcgtcaaaaatagactgacccaagtttttgtattatcagaatatggatggaggatggggactacatgcggaaggcgagagctccatgctcagctcagctctcaactacactgctctaagactacttggtgagggtgctgatgatggaccggatatgtccatgccgaaagcaaggaaatggatacatgaccatggtggtgcaacaatgataccaatcttgggaaaagtctggctctcggtaacaatcatcgtccttgtgccggctccaagtttctattttcagcaagtgattcacaagttagcttgattcgccaaacatttgacgcttcttgttaatatactaagagtaaaaaatatgttcttgttaacaagcaagtcaactgcatgctttcagttatgcagaatacttcaacaagagtaaaagtttgacaaaactatttatgtcgatcgacccgaagcgcatatgaactatatacctgatgctgcaggtacttagggtttttgaatggtcaggtgtaaaccctatgcccccagaattgttccttctgccatccttcgttcctattcaaccaggtacgcagagttcttcgccggcgcgttaagtgttcaaccatgttacgacaattaattacttggttattcaacactaggcatgaaccatattaggaagagcttattttatgagaacaatcatgtggtttctcaaatgcaggacggttgtggagtcacttcagaatggctttcatccccatgtcctatttatatggcaagaagtttgttggcccaataacaaaactcgttttatcattaagggaagagctgcatattcacccctacaaaaagattaactggaagcaagcacgcaaattatgcgcaaaggtgagtccactaactacatagttttaccgtttgttctgcaagtattctacgtgatttcacatttagatttaatttcatatgtagagttctgatctgtcacagttacaatctgtcctcacaactagaataaactgtcagtgaatttcttgctcagttttaaaggattggtgacaacagattatgaattcaaatgcaggaagatgcctatcatccacatacctggctccaagaatgcttgtccgattgcctttacagtttcggtgaaccttttctggcatgttggccagtttcctacatgagacgaaaagctctacgacaaattgctgatttcctgaaatacgaagatgatatttcacggtatatctgcatcggcgccgcgcaaaaggtaaggcttgcggaggtcttatcaaaataattcagtgctgattttcagtgagctgtaaatgaacatcatttcctcacaaagaataaacattattacaggcattatccatgttatgctgttggtctgagaatcccaattcagatgcattcaagcaccacttggctagagttgctgatttcctatggctcggcgaagatgggatgaaagtgcgggtaaattttaatgactcttgtttctgtaatatacagggcgaatatatccttccaatttaaaatgcatctcagtcaaattgaaatttgtgcacaaactaactgatgttacacaacttcattgttcattcaaaaggtgtgtgcaggtcaatcatgggatgtcgctttcgccgtacaagcgatattagcgggtgatgttgcagaggaatttggaagcactctcaagaaagcacaccatttcataaaagcatcccaggtattaattttccagaattgaatcttgttacttatatttctcttgacttaatccctaaccctactaacttaattttgttaagattgtggacaatccttcaggtgacttcgccagaaagtaccgtcacatttctaaaggaggatgggccttccaggttgcggatcagggctggcaagtttcagattgcacagcagaagctcttaaggtgcgatatcaaattagctaaagatgcaaaataaagtggacagaactaaaatggaaacatgcctttcattttgctactacaaagtaatgcatttcattttgtttcagcctttcaggcttctgtaattcctctgtttcaggctctgttactgctctcaaagtttccgtcagatatcgtgggtgatcagatggaaacatgccgcttccatgatgcagtgaacatattattatctttacaggtgtggaaacacatgttgtacttcagattctatattttggtctgtgcatagacaggggcacgtccgtaaagttgggtaaatgttagttttcagtttgatgctagtgaataattcagcactaaggccatatgactggtaacgaaaggcagattcagtacaagcaatgtctactcttatgtgcaagttctgtcaggttgtgtaaaaatgatttgacactagtaggtgtctgcagaatcctaatggtggctatggaacttgggagctagctcgtacacatccttgggtggaggcaagtgtcttgtaaaacacccgcacttcatgctattattatttggagtttaggaaactcaatttaacattgggtaatgagacttttaatatatttttccctttttcagaatttaaacatgacagagatatatgcagacatcatggtggagcatcagtaagaaatctacaacttttcttagcaatagctcttgcatgtgactcaatattaatgcacatacatatcacaggtacgtcgagtgtacctcgtcggtcatccaagcattggccctgtttcggcaaaaataccccgtgcatcgggaagatgaaatagaacagtgcatcaggagagcgacagaattcatcgagaagtcacagaatgaggacggttcatggtcagtctactatcaaaagaattctagtgtttataagtattaacaaggagaaggtgtgttcttgacacattgctacctttcattagctaactaaacaagaaaatggtcacatattgaacttttggtaaaatgctgcaacttctcttgatggagaaattagcaatcgatggtcattaagttattttgcaggtttggatcatggggtgtttgcttcacatatggcacatggtttgctatagggggcctatcagcagttggacagtgttacaataacagcacctacattcggaaggcttgccagtttctattatcgaagcagctaaggaatggtggatggggtgagagtcatctttcatccacaaccaaggtaaacgctgtcgaatgacttcttatgttgatgaacagaaagtgctcacatctttgagaaaatcaagtatcctggcaaagaaaatattctttattctttcttcttccttttttttctttttttttgcggggttattctttcttcatttctttcttcaatataccttgaaacaagatactggcaaaaacacatgcacttcctatggtaactgatcacacatgtcatctcaaattctgaacaggcttacacgaacctagatggggagaaatcacacatagtcaacaccgcatgggcgatgttggcgctaatgaaagctggacaggtgttcacttctctttctctctccaaatgtcaaattccaaagaactaccatggagttaatgcacctttagccccagaataattgaaccaatctatagtataacttctgatcttgggaaaaaaattataggataacttcaaggttcaggttctaattagttccattgaattatgaagtattgaattaattctagcttttcttcgaagttcagggcgaaagagatgcatctcctttgcacaaagcagcaggacttatcatgaacatgcaactcggcaatggtgacttccctcaggaggtaactactgctacgatgtaccatgtgttatacacttatactacaatttgatccaaaatcctatattctgatcttatattcttatcttataatgctataatactataatttgttgttattatgcaggaaatgattggaagtttcttgaagaatggccccttatgttatatggcttatcgcaacatattccccatatgggcccttggagagtatcatagattggtactttgctccgactaa

SEQ ID NO: 8 - DNA, 2280 bp, Triticum aestivum
  source 1..2280: mol_type = other DNA
atgtggaagctcaagattgctgagggtggcccgtggctcacgagcggtaacaatcacgtcggaagagaaacatgggagtttgaccaaaatgacaccggatcaagcgaagagcgggatgcggtcgacgcttcacgggccgaattccagaagaacaggttcaggacaaggcacagctccgatgttttggcccgcatgcagctagcaaaggggaataagttcagccttgaccaacaacagaaaccaaaagatgatgaaactagtggtgatatcaatatagctacggtgtcggagacactgagaagggcactcagatacttctcagccgtacaagcgcatgatgggcactggccaggagattttccggggcctctctttaccacagcaaccatgatcatagttttgtatgtcacggagtcgttaggtactacgctatcgtcagaacaccgcaaggagatctgtcgctatctgtacaaccgacagaatatggatggaggatggggactacatgcggaaggcgagagctccatgctcagctcagctctcaactacactgctctaagactacttggtgagggtgctgatgatggaccggatatgtccatgccgaaagcaaggaaatggatacatgaccatggtggtgcaacaatgataccaatcttgggaaaagtctggctctcggtacttagggtttttgaatggtcaggtgtaaaccctatgcccccagaattgttccttctgccatccttcgttcctattcaaccaggacggttgtggagtcacttcagaatggctttcatccccatgtcctatttatatggcaagaagtttgttggcccaataacaaaactcgttttatcattaagggaagagctgcatattcacccctacaaaaagattaactggaagcaagcacgcaaattatgcgcaaaggaagatgcctatcatccacatacctggctccaagaatgcttgtccgattgcctttacagtttcggtgaaccttttctggcatgttggccagtttcctacatgagacgaaaagctctacgacaaattgctgatttcctgaaatacgaagatgatatttcacggtatatctgcatcggcgccgcgcaaaaggcattatccatgttatgctgttggtctgagaatcccaattcagatgcattcaagcaccacttggctagagttgctgatttcctatggctcggcgaagatgggatgaaagtgcgggtgtgtgcaggtcaatcatgggatgtcgctttcgccgtacaagcgatattagcgggtgatgttgcagaggaatttggaagcactctcaagaaagcacaccatttcataaaagcatcccagattgtggacaatccttcaggtgacttcgccagaaagtaccgtcacatttctaaaggaggatgggccttccaggttgcggatcagggctggcaagtttcagattgcacagcagaagctcttaaggctctgttactgctctcaaagtttccgtcagatatcgtgggtgatcagatggaaacatgccgcttccatgatgcagtgaacatattattatctttacagaatcctaatggtggctatggaacttgggagctagctcgtacacatccttgggtggagaatttaaacatgacagagatatatgcagacatcatggtggagcatcagtacgtcgagtgtacctcgtcggtcatccaagcattggccctgtttcggcaaaaataccccgtgcatcgggaagatgaaatagaacagtgcatcaggagagcgacagaattcatcgagaagtcacagaatgaggacggttcatggtttggatcatggggtgtttgcttcacatatggcacatggtttgctatagggggcctatcagcagttggacagtgttacaataacagcacctacattcggaaggcttgccagtttctattatcgaagcagctaaggaatggtggatggggtgagagtcatctttcatccacaaccaaggcttacacgaacctagatggggagaaatcacacatagtcaacaccgcatgggcgatgttggcgctaatgaaagctggacagggcgaaagagatgcatctcctttgcacaaagcagcaggacttatcatgaacatgcaactcggcaatggtgacttccctcaggaggaaatgattggaagtttcttgaagaatggccccttatgttatatggcttatcgcaacatattccccatatgggcccttggagagtatcatagattggtactttgctccgactaa

SEQ ID NO: 9 - DNA, 4954 bp, Triticum aestivum
  source 1..4954: mol_type = genomic DNA
atgtggaagctcaagatcgccgagggcggcccgtggctcacgagcggcaacaatcacgtcggaagagaaacatgggagtttgaccaaaacgatgccggatcaagcgaagagcgggatgcggtcgacactgcacgggccgaattccagaagaacaggttcaggacaaggcacagctccgatgttttggctcgcatgcaggtaactaaaatgcactgatatgtatgcacctttgcggtctctgatgatatttgaagctttttattataagaaactgagtacatgctctgtttttcgttctgcagctagcaaagaagaataacttcagccttgatcagcagaaaccaaatgatgaagctactgtcgacatcaatgtagctacggtatcagaggcactgagaagggcactcggatacttctccgccatacaagcgcatgatgggcactggccaggagattttccggggccactctttaccacagcaaccatggtaattaagatacatcataattaaaatttctgtttaattgtttgggtcatcaccatcaagttagttatagccaacatttagatgctcatgcacactgtattctcatattcttgcacttccacatgacattcttatgcttattccttcctgctgcagatcatagttttgtatgtcaccgagtcattaggtactacgctgtcatcggagcatcgcaaggagatctgtcgttacttgtacaaccgtcaggtaattaagctgcacattttccaggaaactagtcatggataactaggaagttctgttttccacaatatatattttgactgaaactgtttctctggatgtcttcaaaattttttagaagaaaaatatatcatcaaaaatagactgacccaatttttgtatcatcagaatatagatggaggctggggactacacgcagaaggcgaaagctccatgctcagctcagctctcaactacactgctctaagactacttggtgagggcgctgatgatggaccggatatgtccatgccgaaagctcggaaatggatacatgaccatggtggtgcaacaatgataccaatcttgggaaaagtgtggctctcggtaacaatcatcacccttgtgccggccccaaccttttattttcagcaagtgattcacaagttagcttgattcgccaaacatttcaccgcttcttgttaatatactcggagtaaaaattatgttcttgttaacaagcaagtctgaaactgcatgctttcagttatgcagaattcttcaacaagagtaaaaatttgacgaaactatttatgtcgatcgacccaaagcgcatctgaactgtgtgcatgcctgatgctgcaggtacttggggtttttgaatggtcaggtgtaaaccctatgcccccagaattgttccttctgccatccttcgttcctattcaaccaggtacgcagagttcttcgccggcgcgttaagtgttcaaccatgttacgacagttaattacttggtaggtctgaaccacattaggaagagcttattttatgagaacaatcatgttgtttctcaagtgcaggacggttgtggagtcacttcagaatggctttcatccccatgtcctatttatatggcaagaagtttgttggcccaataacaaaactggttttatcattaagagaagagctgcatattcacccctacaaaaagattaactggaagcaagcgcgcaaattatgcgcaaaggtgagtccactaaactatatagttttaccgcttgttcggcaaatattccacgtgattttacacttagatttaatttcgtatgtagagttctgatctgtcacaattaccatttgtcctcgccaatagaatcaactgtcagtgaatttcttgttcaattttaaaggatcggtgacaacagattatgaattcaaatgcaggaagatgcctatcatccacatacctggctccaagaatgcttgtctgactgcctttacagtttcggtgtaccgtttctggcatgttggccggtttcctacatgagacgaagagctctacgacaaattgccgatttcctgaaatacgaagatgatatttcacggtatatctgcatcggcgccgcgcaaaaggtaaggcttacggaggtcttcacaaaataattcagtgctgggttttcagtgagctgtaaatgaacatcatttcctgacagagaataaacattattacaggcattatccatgttatgctgttggtctgaggatcccaattcagatgcattcaagcgccacttggctagagtcgctgatttcctatggctcggtgaagatggcatgaaagtgcgggtaaattttaataactcttgtttatgtaatatagtccatttatgtatcccagtcaaactgaaatttgtgcacaaactaactgatattacacgacttcattgttcgttcgaaaggtatgtgcgggccaatcatgggatgttgcttttgccgtacaagcgatattagcgtctaatgttgcagaggaatttggaactactctcaggaaagcacatcatttcataaaagcatcccaggtaatatatttttaccagaactgaatcttgttacttatatttatcttgacttaatccctaatcctactaccttaattttgttaagattgtggacaatccttcaggtgacttcgccagaaagtaccgtcacatctctaaaggaggatgggccttccaggttgcagaccagggctggcaggtttcagattgcacagcagaagctctcaaggtgcgataccaaattaactaaagatgcaaaagaaagtgaacaaaactaaaatggaaacatgcctttcattttgctactacaaagtaatgcatttcattttgtttcagcctttcaggcttttgtaattcttcttctgtttcaggctctgttactgctctcgaagtttccgtcagatatcgtgggtgatcagatggaaacatgccgcttccatgatgcggtgaacgtattactatctttacaggtgtgtaaccacatgttgtacttcagcagttccgctacaaatgtacaccaatgccaagcaccgcgcagagtaaaaatgatttgacacttaggttgtatgcagaatcctaatggtggctatggaacttgggagctagctcgtacatatccatggatggaggcaagtgttctggaaacacttgcgctgtgcgctatttttatttggaatttaggcatctcaatttaacatgggtaatgagatcccgcaaaaaaaaacatgggtaatgagacttctaacattttccccttttcagaatttaaacatgacagagatatatgcggacatcatggtggagcatcagtaagatatgtacaacctttcttagcaatagctcttgcatgtgactcgatattaagatgcacacatatcgcaggtacgtcgagtgtacctcatcggtcatccaagcattggccctgttttggcaaaagtaccccgggcatcgggaagatgaaatagaacagtgcatcaggagagcgacagaattcattgagaagttgcagaatgaagacggttcatggtcagtatactatcaacagtgttatagtgtttatattaacatggagaaggtgacacattgctgctgcttgggttgccctttaagttttcacattacactgaccaaatattgataattatagctgatgaaactagaaagaaattaattaaacaaatggcacatattgaacttttggcaaaatgttgcaacttctcttgatggagaaattgataatcaatgatcaataagttgttttgcaggtttggatcatggggtatttgcttcacatatggcacatggtttgctatagagggcctatcagcagttggacagtgttacaataacagcacctacattcggaagggttgccagtttctattatcaaagcagctaaggaatggtggatggggtgagagccatctttcatccacaaccaaggtaaacgctgtcgaatggcttcttatgttgatgaacagaaagtgctcacatctttgagaaaatcaagcatcctgacaaagaaaatattctttattcttctttttatctttttttgaggggttattctttcttacccgcaaaaaaaaagagaggggttattctttcttcatttttttcttcaatataagttgaaacaagatactggcaaaaaagtctgcactttctacggtaactgaccacagatgtcatctcaaaatctaaacaggcatacaccaacctagacggggagaaatcacacatagtcaacaccgcatgggcgatgttggcgcttatgaaagctggacaggtgatcacttctctttctctctccaaaggtttcacacggtcaaattccaaaagactagtattatggtaaagcgcagtatggttaatgcacctttggccccagaatagttgaaccgatctatagtataacttctgatcttggaaaaaattataggagaacttcaaggtgcaggttctaattcagttgaactacgaagtagtatcgaattaatcccagcttttcttcgacttcaggccgaaagagatccgtctcctttgcacgaatctgcaagacttatcatgagcatgcagcttggcaatggtgacttcccacaggaggtaactgctacaattaatgtttttttctttcaaaaatgaggattacccccggcctctccatcgagtttgctacaattaatgtaccatggcgttacacgatttaaaccgaaatcttgaattcttgttttcttaataattctatagtaaaaatgattggagctgactcaatcttgtatattattatgtaggaaatgattggaagtttcttgaaaaatggtcccttgtgttatatggcttatcgcaacatattccccatatgggcccttggagagtatcatagattagtactttgctctggctaa

SEQ ID NO: 10 - DNA, 2274 bp, Triticum aestivum
  source 1..2274: mol_type = other DNA
atgtggaagctcaagatcgccgagggcggcccgtggctcacgagcggcaacaatcacgtcggaagagaaacatgggagtttgaccaaaacgatgccggatcaagcgaagagcgggatgcggtcgacactgcacgggccgaattccagaagaacaggttcaggacaaggcacagctccgatgttttggctcgcatgcagctagcaaagaagaataacttcagccttgatcagcagaaaccaaatgatgaagctactgtcgacatcaatgtagctacggtatcagaggcactgagaagggcactcggatacttctccgccatacaagcgcatgatgggcactggccaggagattttccggggccactctttaccacagcaaccatgatcatagttttgtatgtcaccgagtcattaggtactacgctgtcatcggagcatcgcaaggagatctgtcgttacttgtacaaccgtcagaatatagatggaggctggggactacacgcagaaggcgaaagctccatgctcagctcagctctcaactacactgctctaagactacttggtgagggcgctgatgatggaccggatatgtccatgccgaaagctcggaaatggatacatgaccatggtggtgcaacaatgataccaatcttgggaaaagtgtggctctcggtacttggggtttttgaatggtcaggtgtaaaccctatgcccccagaattgttccttctgccatccttcgttcctattcaaccaggacggttgtggagtcacttcagaatggctttcatccccatgtcctatttatatggcaagaagtttgttggcccaataacaaaactggttttatcattaagagaagagctgcatattcacccctacaaaaagattaactggaagcaagcgcgcaaattatgcgcaaaggaagatgcctatcatccacatacctggctccaagaatgcttgtctgactgcctttacagtttcggtgtaccgtttctggcatgttggccggtttcctacatgagacgaagagctctacgacaaattgccgatttcctgaaatacgaagatgatatttcacggtatatctgcatcggcgccgcgcaaaaggcattatccatgttatgctgttggtctgaggatcccaattcagatgcattcaagcgccacttggctagagtcgctgatttcctatggctcggtgaagatggcatgaaagtgcgggtatgtgcgggccaatcatgggatgttgcttttgccgtacaagcgatattagcgtctaatgttgcagaggaatttggaactactctcaggaaagcacatcatttcataaaagcatcccagattgtggacaatccttcaggtgacttcgccagaaagtaccgtcacatctctaaaggaggatgggccttccaggttgcagaccagggctggcaggtttcagattgcacagcagaagctctcaaggctctgttactgctctcgaagtttccgtcagatatcgtgggtgatcagatggaaacatgccgcttccatgatgcggtgaacgtattactatctttacagaatcctaatggtggctatggaacttgggagctagctcgtacatatccatggatggagaatttaaacatgacagagatatatgcggacatcatggtggagcatcagtacgtcgagtgtacctcatcggtcatccaagcattggccctgttttggcaaaagtaccccgggcatcgggaagatgaaatagaacagtgcatcaggagagcgacagaattcattgagaagttgcagaatgaagacggttcatggtttggatcatggggtatttgcttcacatatggcacatggtttgctatagagggcctatcagcagttggacagtgttacaataacagcacctacattcggaagggttgccagtttctattatcaaagcagctaaggaatggtggatggggtgagagccatctttcatccacaaccaaggcatacaccaacctagacggggagaaatcacacatagtcaacaccgcatgggcgatgttggcgcttatgaaagctggacaggccgaaagagatccgtctcctttgcacgaatctgcaagacttatcatgagcatgcagcttggcaatggtgacttcccacaggaggaaatgattggaagtttcttgaaaaatggtcccttgtgttatatggcttatcgcaacatattccccatatgggcccttggagagtatcatagattagtactttgctctggctaa

SEQ ID NO: 11 - DNA, 4809 bp, Triticum aestivum
  source 1..4809: mol_type = genomic DNA
atgtggaagctcaagatcgccgagggcggcccgtggctcacgagcggcaacaaccacttcggaagagaaacatgggagtttgaccgaaatgacgctggatcaagcgaagagcgggatgcggtcgacgctgcacgggctgaattccagaagaacaggttcaggacaaggcacagctccgatgttttggcccgcatgcaggtaagtaaaatgcactgatatatacacccttgtcttggtggtatctatccaactgaatatttgaagtgttttttgttgtgcagttagtaaaggggaataacttcagccttgaccaacaacagaaaccaaaaggtgatgaaactagtgtcgacatcaacatagctacggtgtcggagacactgaaaagggcactcagatacttctcagccatacaagcgcacgatgggcactggccaggagattttccgggccctctctttaccacagcaaccatggtaattaagatgcatcatacataaacttgatgtttgtttgtttgcttcatcatgaccatcaagttagttatgataaacatttatttagatggttatgcatgctatatattcttgctaactattcacaactttggcgtgacatcgacatcgacatgggagacattctgatgtttgttcattcctgcttgctgcagatcgtagttttgtatgtcacggagtcgttaggtactacgctatcgtcagaacaccgcaaggagatctgtcgctatttgtacaaccgacaggtaaggtgcacgcacatttgccaggaaattaattatgggtaacttttagcaagttctgttttccacaatatatattttcactaaaactgtttctacggatgtcttcaaaatttttacacaagaaaattatatcggaataaatagactgacccaagtttttgtattatcagaatatggatggaggatggggactacatgcggaaggcgagagctccatgctcagctcagctctcaactacgctgctctaagactacttggtgagggtgctgatgatggaccagatatgtccatgccaaaagctaggaaatggatacatgaccatggtggtgcaacaatgataccaatattgggaaaagtgtggctttcggtaacaatcatcctccttgtgccggccccaagtttttattttcagcaatgattcacaagttagcttgattcgccaaacatttcaccacttcttgttaatatactcaaagagtaaaaattatgttcttgttaacaagcaagtctgaaactgcatgctttcagttatgcagaattcttcaacaagagtaaaaatttgacgaaactatttacgtcgatcgacccaaagtgcatctgaactgtatgcctgacgctgcaggtacttggggtttctgaatggtcaggtgtaaaccctatgcccccagaattgttccttctgccatccttcgttcctatccaaccaggtacgcagagttcttcaccggtgcgttaagtgttcaaccatgttactacaattaattacctggttattcaacactaggtatgaaccatattaggaagagcttattttatgagaacaatcatattgtttctcaaatgcaggacggttgtggagtcacttcagaatggctttcatccccatgtcctatttatatggcaagaagtttgttggcccaataacaaaactggttttatcattaagggaagagctgcatattcacccctacaaaaagattaactggaagcaagcgcgcaaattatgcgcaaaggtgagtccactaactttatagtttttaccttttgttctgcgagtattccacgtgatttcacatttggattaatttcttgttcaattttaaaggattatgaattcaaatgcaggaagatgcgtatcatccacatacctggctccaagaatgcttgtccgattgcctttacagtttcggtgaaccttttctggcatgttggccagtttcccacatgagacgaaaagctctacgacaaattgccgatttcctgaaatacgaagatgatatttcacggtatatctgcattggcgctgcgcaaaaggttagacttacggaggtcttcacaaaataattcagtgctgattttcagtgagctgtaaatgaacatcatttccttacaaagaataaaccatattacaggcattatccatgttatgctgttggtctgagaatcccagttcagatgcattcaagcgccacttggctagagtttctgatttcctatggctcggtgaagatggcatgaaagtgcgggtaagttttaaaaattcttctttctgtagtacagaatatatccttccatttagtcaaaattgaaatttgtgcacaaactgatattacatgtcattgttcattcaaaaggtatgtgcaggtcagtcatgggatgttgcttttgctgtacaagcaatattagcgtgtgatgttgcacaggaatttggaactactctgaagaaagcacaccatttcataaaagcatcccaggtaatatattttttctagaactgaattttgttacgtacatttattttgactgaaaccctaactctactaacttacaatttttttttttaagattgtcagcaatcctaccggtgacttcagcagaaagtaccgtcacatctctaaaggaggatgggccttccaggttgcagaccagggctggcaggtttcagactgcacagcagaagctctcaaggtgtgatatcaaattaattaaggatgcaaaagaaagtgaacagaactaaaatggaaacatgcctttcattttgctactacaaagtaatgcatttcattttgtttcagcctttgaggcttttgtaactcctctgtttcaggctctgttactgctctcaaagtttccgtcagagatcgtgggtgatcagatggaaacatgccgcttccatgatgcagtgaacatattattatctttacaggtgtggaaccacaacttgtactttagattctatattttggtctatgcatacacaggggcatgtccataaagttgggtaaatgttagttttcagttactccctccgttcggaattcttctagatacatccatttccaagacaagtaattccgaacggagggagtagatgctagtgaataattcagcacaaggccgtatggctggtaacgaaaggcagattcagtgcgatcagtgtctactcttatgtgcatgattctgatatgcaatctttacttgaagcaagtctgtcaggttgtgtaaaaatgatttgacactaggttgtacgcagaatcctaatggtggctatggaacttgggagctagctcgtacatatccatggatggaggcaagtgctctgcaaacacctgcactccatgttgtttttatttggagtttaggaaaatcaatttaacattgggtaatgagacctttaatatatttttttcttttccagaatttaaacatgacagagatatatgcagacatcatggtggagcatcagtaagaaatctacaatctttcttagcaatagctcttgcatgtgactcgatattaagatgcatacatgtcgcaggtacgtcgagtgtacctcgtcggtcatccaagcattggccctgtttcggcaaaaataccccgggcatcgggaagatgaagtagaacaatgcatcaggagagcgacagaattcatcgagaagttacagaatgaggacggttcatggtcagtacactatcaacagttttatagtgtttatattaagtttattaaacaaggagaaggtgtgctgttgacatattgatcaacaagttgttttgcaggttcggatcatggggtatttgcttcacatacggcacgtggtttgctatagagggcctatcggcagttggacagtgttacaataatagcacctacatccggaagggttgccagtttctattatcaaagcagctaaggaatggtggatggggtgagagtcatctttcatccacaaccaaggtaaacgctatcgaatggcttcttatgttgatgaacagaaagtactcacatcttagagaaaatcaagcataccgacaaagaaatttttaattattcttgtttcaatttaccttgaattctgcactttctacggtaaccgatcatacgtgtcatctcgaattctaaacaggcatacacgaacctagacggggagaaatcacatgtagtcaacaccgcatgggcaatgttggcactaatgaaagctggacaggtgaccacttctctttctctctccaaaggtttcacatggtcaaagtccaaaagactactattatgttaatgcgcagtatcgtaaatgcacgaaaggtttccccagaataactgaaccgatctatagtataacttctgatcttggaaaaaattataggagaacttcaaggtgcaggttctaattcaattgaactacgaagtactgtcgaattaattccagcttttcttcgacatcaggccgaacgagatccgtctcctttgcacgaagctgcaagacttatcatgagcatgcaacttggcaatggtgacttcccacaggaggtaactaactgctacagttaatgtaccatgcgctacacgatttaaaccgaaatcttgaattcttgttttcttaataatgctataataaacatgattggagctgactcaatcttgtatattattatgtaggaaatgattggaagtttcttgaaaaatggtcccttgtgttatatggcttatcgcaacatattccccatatgggcccttggagagtatcatagactagtactttgctctggctaa

SEQ ID NO: 12 - DNA, 2280 bp, Triticum aestivum
  source 1..2280: mol_type = other DNA
atgtggaagctcaagatcgccgagggcggcccgtggctcacgagcggcaacaaccacttcggaagagaaacatgggagtttgaccgaaatgacgctggatcaagcgaagagcgggatgcggtcgacgctgcacgggctgaattccagaagaacaggttcaggacaaggcacagctccgatgttttggcccgcatgcagttagtaaaggggaataacttcagccttgaccaacaacagaaaccaaaaggtgatgaaactagtgtcgacatcaacatagctacggtgtcggagacactgaaaagggcactcagatacttctcagccatacaagcgcacgatgggcactggccaggagattttccgggccctctctttaccacagcaaccatgatcgtagttttgtatgtcacggagtcgttaggtactacgctatcgtcagaacaccgcaaggagatctgtcgctatttgtacaaccgacagaatatggatggaggatggggactacatgcggaaggcgagagctccatgctcagctcagctctcaactacgctgctctaagactacttggtgagggtgctgatgatggaccagatatgtccatgccaaaagctaggaaatggatacatgaccatggtggtgcaacaatgataccaatattgggaaaagtgtggctttcggtacttggggtttctgaatggtcaggtgtaaaccctatgcccccagaattgttccttctgccatccttcgttcctatccaaccaggacggttgtggagtcacttcagaatggctttcatccccatgtcctatttatatggcaagaagtttgttggcccaataacaaaactggttttatcattaagggaagagctgcatattcacccctacaaaaagattaactggaagcaagcgcgcaaattatgcgcaaaggaagatgcgtatcatccacatacctggctccaagaatgcttgtccgattgcctttacagtttcggtgaaccttttctggcatgttggccagtttcccacatgagacgaaaagctctacgacaaattgccgatttcctgaaatacgaagatgatatttcacggtatatctgcattggcgctgcgcaaaaggcattatccatgttatgctgttggtctgagaatcccagttcagatgcattcaagcgccacttggctagagtttctgatttcctatggctcggtgaagatggcatgaaagtgcgggtatgtgcaggtcagtcatgggatgttgcttttgctgtacaagcaatattagcgtgtgatgttgcacaggaatttggaactactctgaagaaagcacaccatttcataaaagcatcccagattgtcagcaatcctaccggtgacttcagcagaaagtaccgtcacatctctaaaggaggatgggccttccaggttgcagaccagggctggcaggtttcagactgcacagcagaagctctcaaggctctgttactgctctcaaagtttccgtcagagatcgtgggtgatcagatggaaacatgccgcttccatgatgcagtgaacatattattatctttacagaatcctaatggtggctatggaacttgggagctagctcgtacatatccatggatggagaatttaaacatgacagagatatatgcagacatcatggtggagcatcagtacgtcgagtgtacctcgtcggtcatccaagcattggccctgtttcggcaaaaataccccgggcatcgggaagatgaagtagaacaatgcatcaggagagcgacagaattcatcgagaagttacagaatgaggacggttcatggttcggatcatggggtatttgcttcacatacggcacgtggtttgctatagagggcctatcggcagttggacagtgttacaataatagcacctacatccggaagggttgccagtttctattatcaaagcagctaaggaatggtggatggggtgagagtcatctttcatccacaaccaaggcatacacgaacctagacggggagaaatcacatgtagtcaacaccgcatgggcaatgttggcactaatgaaagctggacaggccgaacgagatccgtctcctttgcacgaagctgcaagacttatcatgagcatgcaacttggcaatggtgacttcccacaggaggaaatgattggaagtttcttgaaaaatggtcccttgtgttatatggcttatcgcaacatattccccatatgggcccttggagagtatcatagactagtactttgctctggctaa

SEQ ID NO: 13 - DNA, 30 bp, synthetic construct
  source 1..30: mol_type = other DNA
atgtggaagctcaagattgctgagggtggc

SEQ ID NO: 14 - DNA, 30 bp, synthetic construct
  source 1..30: mol_type = other DNA
ttagccagagcaaagtactaatctatgata

SEQ ID NO: 15 - DNA, 30 bp, synthetic construct
  source 1..30: mol_type = other DNA
atgtggaagctcaagatcgccgagggcggc

SEQ ID NO: 16 - DNA, 30 bp, synthetic construct; identical to SEQ ID NO: 14
  source 1..30: mol_type = other DNA

SEQ ID NO: 17 - DNA, 30 bp, synthetic construct; identical to SEQ ID NO: 15
  source 1..30: mol_type = other DNA

SEQ ID NO: 18 - DNA, 30 bp, synthetic construct
  source 1..30: mol_type = other DNA
ttagccagagcaaagtactagtctatgata

SEQ ID NO: 19 - DNA, 18 bp, synthetic construct
  source 1..18: mol_type = other DNA
tggctcacgagcggcaac

SEQ ID NO: 20 - DNA, 21 bp, synthetic construct
  source 1..21: mol_type = other DNA
aatctcctggccagtgcccat

SEQ ID NO: 21 - DNA, 20 bp, synthetic construct
  source 1..20: mol_type = other DNA
ctgagaaacggctaccacat

SEQ ID NO: 22 - DNA, 19 bp, synthetic construct
  source 1..19: mol_type = other DNA
cccaaggtccaactacgag

SEQ ID NO: 23 - DNA, 20 bp, synthetic construct
  source 1..20: mol_type = other DNA
atgatgaaactagtggtgat

SEQ ID NO: 24 - DNA, 20 bp, synthetic construct
  source 1..20: mol_type = other DNA
tccttgcggtgttctgacga

SEQ ID NO: 25 - DNA, 25 bp, synthetic construct
  source 1..25: mol_type = other DNA
cttcagccttgatcagcagaaacca

SEQ ID NO: 26 - DNA, 25 bp, synthetic construct
  source 1..25: mol_type = other DNA
aatgactcggtgacatacaaaacta

SEQ ID NO: 27 - DNA, 24 bp, synthetic construct
  source 1..24: mol_type = other DNA
cagcaatcctaccggtgacttcag

SEQ ID NO: 28 - DNA, 25 bp, synthetic construct
  source 1..25: mol_type = other DNA
gacggaaactttgagagcagtaaca

SEQ ID NO: 29 - DNA, 25 bp, synthetic construct
  source 1..25: mol_type = other DNA
catatgtagagttctgatctgtcac

SEQ ID NO: 30 - DNA, 26 bp, synthetic construct
  source 1..26: mol_type = other DNA
agttcaatatgtgaccattttcttgt

SEQ ID NO: 31 - DNA, 20 bp, synthetic construct
  source 1..20: mol_type = other DNA
cagtttgatgctagtgaata

SEQ ID NO: 32 - DNA, 24 bp, synthetic construct
  source 1..24: mol_type = other DNA
atctgaccacttaaatcctccttc

SEQ ID NO: 33 - DNA, 24 bp, synthetic construct
  source 1..24: mol_type = other DNA
ggaagtgcaagaatatgagaatac

SEQ ID NO: 34 - DNA, 20 bp, synthetic construct
  source 1..20: mol_type = other DNA
tcatcagagaccgcaaaggt

SEQ ID NO: 35 - DNA, 28 bp, synthetic construct
  source 1..28: mol_type = other DNA
ggtggtatctatccaactgaatatttga

SEQ ID NO: 36 - DNA, 28 bp, synthetic construct
  source 1..28: mol_type = other DNA
ttgaacaagaaattaatccaaatgtgaa

SEQ ID NO: 37 - DNA, 21 bp, synthetic construct
  source 1..21: mol_type = other DNA
tctgaactgtatgcctgacgc

(19) United States
(12) Patent Application Publication
(10) Pub. No.: US 2025/0380655 A1
(43) Pub. Date: Dec. 18, 2025

(54) METHOD FOR ACQUIRING HUMIDITY-SENSITIVE MALE-STERILE TRITICUM AESTIVUM L.

(71) Applicant: ANHUI KAITAI AGRICULTURE AND ANIMAL HUSBANDRY TECHNOLOGY CO., LTD., Hefei (CN)

(72) Inventors: Xiaoquan QI, Beijing (CN); Xingchen XIONG, Beijing (CN); Changfeng GUO, Beijing (CN); Yingchun ZHANG, Beijing (CN); Bo SONG, Beijing (CN)

(73) Assignee: ANHUI KAITAI AGRICULTURE AND ANIMAL HUSBANDRY TECHNOLOGY CO., LTD., Hefei (CN)

(21) Appl. No.: 19/157,392

(22) PCT Filed: Jul. 30, 2024
(86) PCT No.: PCT/CN2024/108509

§ 371 Date: Aug. 18, 2025

(30) Foreign Application Priority Data
Aug. 10, 2023 (CN) 202311006149.6

(51) Int. Cl. A01H 1/04; A01H 1/02; A01H 1/06; C12Q 1/6895

(52) U.S. Cl. CPC A01H 1/045; A01H 1/022; A01H 1/06; C12Q 1/6895; C12Q 2600/13

(57) ABSTRACT

A method for acquiring humidity-sensitive male-sterile Triticum aestivum L includes: with Triticum aestivum L. as a donor plant, mutating a 1595th nucleotide of a coding sequence of a TaGMS-A gene from G to A, mutating a 6th nucleotide of a coding sequence of a TaGMS-B gene from G to A, and mutating a 791st nucleotide of a coding sequence of a TaGMS-D gene from C to T to produce the humidity-sensitive male-sterile Triticum aestivum L. The fertility of the humidity-sensitive male-sterile Triticum aestivum L. can be restored by moisturizing ears during a flowering period. Therefore, the humidity-sensitive male-sterile Triticum aestivum L. has a promising application prospect in the crossbreeding of Triticum aestivum L.

### CROSS REFERENCE TO THE RELATED APPLICATIONS

This application is the national phase entry of International Application No. PCT/CN2024/108509, filed on Jul. 30, 2024, which is based upon and claims priority to Chinese Patent Application No. 202311006149.6, filed on Aug. 10, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBJC038-PKG-LD_SequenceListing.xml, created on May 26, 2025, and is 76,409 bytes in size.

### TECHNICAL FIELD

The present disclosure relates to the crossbreeding of Triticum aestivum L., and specifically relates to a method for acquiring humidity-sensitive male-sterile Triticum aestivum L.

### BACKGROUND

Many predictions indicate that the world population will exceed 9 billion by 2050. Accordingly, the Food and Agriculture Organization of the United Nations (2005) has set a goal of increasing the grain output by 60% or more. Triticum aestivum L. is an important grain crop and can provide about 20% of the energy and proteins required by the human in the food. To achieve the increase of 60% in the global grain output, the output per hectare of land must rise from the original 1% to 1.6%. However, the genetic improvements for disease resistance, biotic stress, and abiotic stress of crops can only lead to slight growth in the crop yield. Therefore, conventional breeding approaches must be transformed to achieve substantial growth in grain production. Crossbreeding is the most promising method to achieve the crop yield improvement. Crossbreeding can steadily increase the crop output by 10% or more.

An effective crossing system is a prerequisite for the efficient production of hybrid seeds, especially for predominantly self-pollinating crops such as Oryza sativa L. and Triticum aestivum L. Several methods have been employed to produce hybrid seeds in crops, including physical emasculation, emasculation with chemical hybridization agents (CHAs), and male plant sterility. Male sterility includes genic male sterility (GMS) and cytoplasmic male sterility (CMS). GMS typically results from mutations of nuclear genes, while CMS is caused by alterations in cytoplasmic DNA. For hybrid seed production, both physical and chemical emasculation often require a lot of labor input, resulting in high cost and significant resource consumption. Moreover, it is challenging to guarantee seed purity. The production of CMS lines heavily depends on the maintainer lines with specific genotypes, and the fertility restoration requires dedicated restorer genes. As a result, there are limited breeding resources. GMS is an ideal sterile line for hybrid seed production. GMS can be used directly for hybrid seed production. However, the self-propagation of sterile lines can be achieved by the seed production technology (SPT) based on genetic engineering and molecular manipulation. Therefore, cloning additional GMS genes of Triticum aestivum L. and establishing corresponding male-sterile materials are critical for achieving hybrid seed production. Currently, there are still insufficient GMS-related genetic resources of Triticum aestivum L. Moreover, the cloned gene-mediated sterility traits all are manifested as the malformation of anther development and the failed development or abortion of pollen grains. There are few reports on the male sterility caused by dehydration of mature pollen grains, and there are no related male-sterile materials.

### SUMMARY

The present disclosure is intended to provide a method for acquiring humidity-sensitive male-sterile Triticum aestivum L., humidity-sensitive male-sterile Triticum aestivum L. acquired by the method, and a method for restoring or improving fertility of the humidity-sensitive male-sterile Triticum aestivum L., so as to promote the crossbreeding of Triticum aestivum L.

The present disclosure provides a method for acquiring humidity-sensitive male-sterile Triticum aestivum L., including:
with Triticum aestivum L. as a donor plant, mutating a 1595th nucleotide of a coding sequence of a TaGMS-A gene from G to A, mutating a 6th nucleotide of a coding sequence of a TaGMS-B gene from G to A, and mutating a 791st nucleotide of a coding sequence of a TaGMS-gene from C to T to produce the humidity-sensitive male-sterile Triticum aestivum L.,
where the coding sequence of the TaGMS-A gene is shown in SEQ ID NO: 8, or has an identity of 90% or more with a nucleotide sequence shown in SEQ ID NO: 8;
the coding sequence of the TaGMS-B gene is shown in SEQ ID NO: 10, or has an identity of 90% or more with a nucleotide sequence shown in SEQ ID NO: 10; and
the coding sequence of the TaGMS-D gene is shown in SEQ ID NO: 12, or has an identity of 90% or more with a nucleotide sequence shown in SEQ ID NO: 12.

In the above method, the Triticum aestivum L. can be any suitable Triticum aestivum L. variety, such as Xichang 76-9 or Chinese Spring (CS).

The present disclosure also provides a method for acquiring humidity-sensitive male-sterile Triticum aestivum L., including.
subjecting first Triticum aestivum L. with an accession number of CGMCC No. 45692, second Triticum aestivum L. with an accession number of CGMCC No. 45693, and third Triticum aestivum L. with an accession number of CGMCC No. 45694 to triallelic crossing to produce progeny plants, and
selecting a plant in which a 1595th nucleotide of a coding sequence of a TaGMS-A gene is A, a 6th nucleotide of a coding sequence of a TaGMS-B gene is A, and a 791st nucleotide of a coding sequence of a TaGMS-D gene is T from the progeny plants to obtain the humidity-sensitive male-sterile Triticum aestivum L.,
where the coding sequence of the TaGMS-A gene is shown in SEQ ID NO: 8, or has an identity of 90% or more with a nucleotide sequence shown in SEQ ID NO: 8;
the coding sequence of the TaGMS-B gene is shown in SEQ ID NO: 10, or has an identity of 90% or more with a nucleotide sequence shown in SEQ ID NO: 10; and
the coding sequence of the TaGMS-D gene is shown in SEQ ID NO: 12, or has an identity of 90% or more with a nucleotide sequence shown in SEQ ID NO: 12.

The term “triallelic crossing” refers to a breeding method in which first and second parental plants are crossed to produce F1 (a first-generation hybrid) and then F1 is crossed with a third parental plant, which can be represented as (A×B)×C.

In the first Triticum aestivum L., the 1595th nucleotide of the coding sequence of the TaGMS-A gene is A. In the second Triticum aestivum L., the 6th nucleotide of the coding sequence of the TaGMS-B gene is A. In the third Triticum aestivum L., the 791st nucleotide of the coding sequence of the TaGMS-D gene is T.

In order to acquire the humidity-sensitive male-sterile Triticum aestivum L., the first Triticum aestivum L. can be crossed with the second Triticum aestivum L. to produce F1 plants. A plant in which the 1595th nucleotide of the coding sequence of the TaGMS-A gene is A and the 6th nucleotide of the coding sequence of the TaGMS-B gene is A is selected from the F1 plants to obtain a double-mutant plant. The double-mutant plant is crossed with the third Triticum aestivum L. to produce F2 plants. A plant in which the 1595th nucleotide of the coding sequence of the TaGMS-A gene is A, the 6th nucleotide of the coding sequence of the TaGMS-B gene is A, and the 791st nucleotide of the coding sequence of the TaGMS-D gene is Tis selected from the F2 plants to obtain a triple-mutant plant, which is the humidity-sensitive male-sterile Triticum aestivum L. Similarly, the first Triticum aestivum L. can be crossed with the third Triticum aestivum L., and screening is conducted to obtain a double-mutant plant. Then the double-mutant plant can be crossed with the second Triticum aestivum L., and screening is conducted to obtain a triple-mutant plant. Similarly, the second Triticum aestivum L. can be crossed with the third Triticum aestivum L., and screening is conducted to obtain a double-mutant plant. Then the double-mutant plant can be crossed with the first Triticum aestivum L., and screening is conducted to obtain a triple-mutant plant.

In the above method, a dehydration rate of pollen of the humidity-sensitive male-sterile Triticum aestivum L. is higher than a dehydration rate of pollen of corresponding wild-type (WT) Triticum aestivum L.

Humidity-sensitive male-sterile Triticum aestivum L. acquired by the above method also falls within the scope of the present disclosure.

The humidity-sensitive male-sterile Triticum aestivum L. can be Triticum aestivum L. with an accession number of CGMCC No. 45691.

A use of the humidity-sensitive male-sterile Triticum aestivum L. with an accession number of CGMCC No. 45691 in crossbreeding of Triticum aestivum L. also falls within the scope of the present disclosure.

In the above use, a method for restoring or improving fertility of the humidity-sensitive male-sterile Triticum aestivum L. is as follows. during a flowering period of the humidity-sensitive male-sterile Triticum aestivum L., maintaining a humidity in an environment for an ear of the humidity-sensitive male-sterile Triticum aestivum L. at 90% or more.

In the above use, a method for maintaining the humidity in the environment for the ear can be as follows: wrapping the entire ear of the humidity-sensitive male-sterile Triticum aestivum L.

In the above use, a method for the wrapping can be as follows: before pollen shedding and after heading of the humidity-sensitive male-sterile Triticum aestivum L., covering the entire ear with a transparent plastic bag or wrapping the entire ear with a transparent plastic film until the pollen shedding is completed. The transparent plastic film can be a plastic wrap.

The present disclosure also provides a method for restoring or improving fertility of Triticum aestivum L., including: during a flowering period of the Triticum aestivum L., maintaining a humidity in an environment for an ear of the Triticum aestivum L. at 90% or more, where the Triticum aestivum L. is humidity-sensitive male-sterile Triticum aestivum L. in which a 1595th nucleotide of a coding sequence of a TaGMS-A gene is A, a 6th nucleotide of a coding sequence of a TaGMS-B gene is A, and a 791st nucleotide of a coding sequence of a TaGMS-D gene is T;
the coding sequence of the TaGMS-A gene is shown in SEQ ID NO: 8, or has an identity of 90% or more with a nucleotide sequence shown in SEQ ID NO: 8;
the coding sequence of the TaGMS-B gene is shown in SEQ ID NO: 10, or has an identity of 90% or more with a nucleotide sequence shown in SEQ ID NO: 10; and
the coding sequence of the TaGMS-D gene is shown in SEQ ID NO: 12, or has an identity of 90% or more with a nucleotide sequence shown in SEQ ID NO: 12.

In the above method, the humidity-sensitive male-sterile Triticum aestivum L. can be Triticum aestivum L. with an accession number of CGMCC No. 45691.

In the above method, a method for maintaining the humidity in the environment for the ear can be as follows: wrapping the entire ear of the humidity-sensitive male-sterile Triticum aestivum L.

In the above method, a method for the wrapping can be as follows: before pollen shedding and after heading of the humidity-sensitive male-sterile Triticum aestivum L., covering the entire ear with a transparent plastic bag or wrapping the entire ear with a transparent plastic film until the pollen shedding is completed. The transparent plastic film can be a plastic wrap.

The present disclosure also provides a humidity-sensitive male-sterile Triticum aestivum L. variety or germplasm with an accession number of CGMCC No. 45691.

In the present disclosure, single-mutant plants S2034, S259, and S924 are constructed by a sodium azide (NaN3) mutagenesis technology with Triticum aestivum L. Xichang 76-9 as a donor plant. In S2034, a 1595th nucleotide of a coding sequence of a TaGMS-A gene changes from G to A. In S259, a 6th nucleotide of a coding sequence of a TaGMS-B gene changes from G to A. In S924, a 791st nucleotide of a coding sequence of a TaGMS-D gene changes from C to T. S2034, S259, and S924 are subjected to triallelic crossing to produce a triple-mutant plant with a genome in which the above three gene loci all change correspondingly. The triple-mutant plant is denoted as tagms-aabbdd. Experiments show that pollen grains of tagms-aabbdd are completely shriveled 60 s after being released from an anther under natural conditions (RH=30% to 60%, Tm=26° C. to 31° C.) (FIG. 2), and thus cannot undergo normal pollination, resulting in complete sterility or almost sterility of the plant (FIGS. 3A-3B). If the entire ear is covered with a transparent plastic bag before pollen shedding and after heading of the tagms-aabbdd plant to make a humidity in an environment for the ear not less than 90% until the pollen shedding is completed, the fertility of tagms-aabbdd can be restored, and a number of seeds per ear can reach 25.3±3.27 (FIGS. 4A-4B). Therefore, the tagms-aabbdd mutant is humidity-sensitive male-sterile Triticum aestivum L., which has a promising application prospect in the crossbreeding of Triticum aestivum L.

Patent deposit information of the Triticum aestivum L. tagms mutant provided by the present disclosure is as follows:

| Biological | tagms1/tapts1- | S2034 | S259 | S924 |
|---|---|---|---|---|
| material: | aabbdd | | | |
| Taxonomic | Triticum aestivum | | | |
| name | | | | |
| Accession | CGMCC No. | CGMCC No. | CGMCC No. | CGMCC No. |
| number | 45691 | 45692 | 45693 | 45694 |

| | |
|---|---|
| Deposit | Jul. 10, 2023 |
| date | |
| Deposit | China General Microbiological Culture Collection Center (CGMCC) |
| unit | |
| Deposit | Institute of Microbiology of the Chinese Academy of Sciences, No. 1, |
| address | Beichen West Road, Chaoyang District, Beijing 100101, China. |

The biological material tagms1/tapts-aabbdd is the triple-mutant plant tagms-aabbdd described herein. The biological materials S2034, S259, and S924 are the single-mutant plants tagms-aaBBDD, tagms-AAbbDD, and tagms-AABBdd described herein, respectively.

### BRIEF DESCRIPTION OF THE DRAWINGS

FIGS. 1A-1B show the expression pattern analysis of TaGMS-A/B/D genes in Triticum aestivum L., where FIG. 1A shows the semi-quantitative analysis of TaGMS-A/B/D genes in different tissues (roots, stems, leaves, and anthers) of Triticum aestivum L. Xichang 76-9, with 18S RNA as an internal reference gene; and FIG. 1B shows the relative expression level analysis of TaGMS-A/B/D) genes in anthers of Triticum aestivum L. Xichang 76-9;

FIG. 2 shows the observation results of morphologies of pollen grains of different mutants of Triticum aestivum L. tagms, where WT represents Triticum aestivum L. Xichang 76-9, tagms-aaBBDD, tagms-AAbbDD, and tagms-AABBdd represent single-mutant plants, and tagms-aabbDD, tagms-aaBBdd, and tagms-AAbbdd represent double-mutant plants; tagms-aabbdd represent triple-mutant plants; 0 s indicates 0 s after pollen grains are released from an anther (namely, immediately upon the detachment) and 60 s indicates 60 s after pollen grains are released from an anther; and in an environment for observing morphologies of pollen grains, a relative humidity is 40% to 55% and a temperature is 26° C. to 31° C.;

FIGS. 3A-3B show the natural ear seed setting analysis of different mutants of Triticum aestivum L. tagms, where FIG. 3A shows the observation results of ear morphologies and seed numbers of Triticum aestivum L. Xichang 76-9 (WT) and mutants (tagms-aaBBDD, tagms-AAbbDD, tagms-AABBdd, tagms-aabbDD, tagms-aaBBdd, tagms-AAbbdd, and tagms-aabbdd) (scale=1.25 cm), where tagms-aabbdd does not undergo seed setting at all; FIG. 3B shows the statistical analysis of natural ear seed setting of WT and mutants (tagms-aaBBDD, tagms-AAbbDD, tagms-AABBdd, tagms-aabbDD, tagms-aaBBdd, tagms-AAbbdd, and tagms-aabbdd), 30 ears are counted for each plant, and results are expressed as mean+SD; and *** representsP<0.001 according to one-way analysis of variance; and

FIGS. 4A-4B show the seed setting conditions of Triticum aestivum L. ears after a moisturizing treatment, where FIG. 4A shows the ear morphologies and seed numbers of Triticum aestivum L. Xichang 76-9 (WT) and tagms-aabbdd with ears moisturized (humidity >90%) (scale=1.25 cm), FIG. 4B shows the statistical results of numbers of seeds per ear of WT and tagms-aabbdd after the moisturizing treatment; and 10 ears are counted for each plant, and results are expressed as mean+SD.

### DETAILED DESCRIPTION OF THE EMBODIMENTS

The present disclosure is further described below in conjunction with embodiments. It should be understood that the following embodiments are merely intended to explain and illustrate the present disclosure, and do not limit the scope of the present disclosure in any way.

The Triticum aestivum L. “Xichang 76-9” adopted in the following embodiments is a Triticum aestivum L. line selectively bred by the Academy of Agricultural Sciences, Liangshan Yi Autonomous Region, which is an extra-early-maturing, ear-heavy, and high-yielding spring wheat line with stripe rust resistance, powdery mildew resistance, and low susceptibility to leaf rust.

Unless otherwise specified, the reagents adopted in the following embodiments all are the conventional reagents in the art, and can be commercially purchased or prepared in accordance with the conventional methods in the art. Unless otherwise specified, the experimental methods and conditions adopted all are the conventional experimental methods and conditions in the art, and can be referred to relevant experimental manuals, well-known literature, or manufacturer's instructions. Unless otherwise specified, three replicates are set for each of the quantitative tests in the following embodiments, and results are averaged. Unless otherwise defined, all technical and scientific terms used herein have the same meanings as commonly understood by those of ordinary skill in the art to which the present disclosure belongs.

### Example 1. Cloning of Genes TaGMS Associated with Humidity-Sensitive Male Sterility in Triticum aestivum L.

According to the reference genome file IWGSC RefSeq v1.1 for the common hexaploid Triticum aestivum L. CS published by the International Wheat Genome Sequencing Consortium (IWGSC), the inventors found the genes associated with humidity-sensitive male sterility in Triticum aestivum L.: TraesCS7A02G003500, TraesCS4A02G495100, and TraesCS7D02G004000, which were named TaGMS-A, TaGMS-B, and TaGMS-D, respectively.

### Gene Amplification

With CS TaGMS-A/B/D as templates, amplification primers were designed for the full-length genes. Names and sequences of the primers were as follows:

| Primer | Primer sequence | Amplified | Product |
|---|---|---|---|
| name | (5′-3′) | gene | size |
| TaGMS- | ATGTGGAAGCTCAAG | TaGMS-A | 4826 bp |
| A-F | ATTGCTGAGGGTGGC | | |
| | (SEQ ID NO: 13) | | |
| TaGMS- | TTAGCCAGAGCAAAG | | |
| A-R | TACTAATCTATGATA | | |
| | (SEQ ID NO: 14) | | |
| TaGMS- | ATGTGGAAGCTCAAG | TaGMS-B | 4956 bp |
| B-F | ATCGCCGAGGGCGGC | | |
| | (SEQ ID NO: 15) | | |
| TaGMS- | TTAGCCAGAGCAAAG | | |
| B-R | TACTAATCTATGATA | | |
| | (SEQ ID NO: 16) | | |
| TaGMS- | ATGTGGAAGCTCAAG | TaGMS-D | 4809 bp |
| D-F | ATCGCCGAGGGCGGC | | |
| | (SEQ ID NO: 17) | | |
| TaGMS- | TTAGCCAGAGCAAAG | | |
| D-R | TACTAGTCTATGATA | | |
| | (SEQ ID NO: 18) | | |

Genomic DNA of Triticum aestivum L. “Xichang 76-9” was extracted through the following steps:

1) A steel ball was added to a 2 mL centrifuge tube, and 200 mg of Triticum aestivum L. leaves was collected and added to the centrifuge tube, quickly frozen with liquid nitrogen, and then ground for 30 s with a ball mill at an amplitude of 30 Hz to produce a powder. Then 1 mL of a 2×CTAB solution (100 mM Tris-HCl with pH 8.0, 20 mM EDTA with pH 8.0, 1.4 M NaCl, 2% CTAB, 2% polyvinylpyrrolidone, and 2% β-mercaptoethanol) was added, and shaking was fully conducted for thorough mixing. Incubation was conducted in a 65° C. oven for 45 min to 1 h, during which slow shaking was conducted a few times every 15 min.

2) After lysis was completed, a sample was taken out, cooled to room temperature, and centrifuged at 12,000 rpm for 10 min. A resulting supernatant (400 μL to 600 μL) was transferred to a new 2 mL centrifuge tube, an equal volume of a chloroform-isoamyl alcohol (v: v=24:1) extraction buffer was added, and slow extraction was allowed for 20 min.

3) Centrifugation was conducted at 4° C. and 12,000 rpm for 10 min. 600 μL of a resulting supernatant was taken and added to a new 1.5 mL centrifuge tube, a ⅔ volume of isopropyl alcohol (the isopropyl alcohol was pre-cooled in a −20° C. freezer for 20 min in advance), and thorough mixing was conducted.

4) Centrifugation was conducted at room temperature and 12,000 rpm for 5 min to 10 min. A resulting supernatant was discarded, and a resulting precipitate was washed twice with 70% ethanol.

5) A resulting precipitate was air-dried at room temperature, and 20 μL of a TE buffer of 200 ng/ml RNaseA was added for dissolution. Incubation was conducted at 37° C. for 30 min to remove RNA to obtain the genomic DNA, which was stored in a −20° C. freezer for later use.

With the extracted genomic DNA as a template, the full-length TaGMS-A, TaGMS-B, and TaGMS-D genes each were amplified using the above primers through polymerase chain reaction (PCR). PCR was carried out using a KOD FX high-fidelity enzyme (Toyobo (Shanghai) biotech Co., Ltd., KFX-101). A reaction system was as follows:

| | |
|---|---|
| 2 × PCR buffer for KOD FX | 25 μl |
| 2 mM dNTPs | 10 μl |
| Forward primer (10 mM) | 1.5 μl |
| Reverse primer (10 mM) | 1.5 μl |
| Genomic DNA of Xichang 76-9 | 1 μl |
| KOD FX | 1 μl |
| ddH₂O | 10 μl |

A reaction program was as follows: pre-denaturation (94° C., 2 min), 1 cycle; and denaturation (98° C., 10 s) and extension (68° C., 5 min), 30 cycles.

### Vector Construction

The amplified TaGMS-A, TaGMS-B, and TaGMS-D genes each were ligated to a pLB-Simple vector with a pLB Zero Background Fast Cloning Kit (TIANGEN (Beijing) Biotech Co., Ltd., Catalog No.: VT206). A ligation system was as follows:

| | |
|---|---|
| TaGMS gene amplification product | 2 μl |
| pLB-Simple Vector | 1 μl |
| 2 × Reaction Solution | 5 μl |
| T4 DNA Ligase | 1 μl |
| ddH₂O | 1 μl |

A centrifuge tube was flicked to make a reaction solution thoroughly mixed, and centrifuged briefly for 3 s to 5 s. A resulting mixed solution was placed at room temperature to allow a reaction for 30 min. 50 μL of a DH5a competent cell suspension prepared in advance was taken and placed in an ice bath. 5 μL of a ligation product was added to the competent cell suspension (which was just to be thawed), and thorough mixing was conducted through flicking. Standing was allowed in an ice bath for 30 min. A centrifuge tube was placed in a 42° C. water bath to allow a heat shock for 90 s, and then immediately transferred to an ice bath to make cells cooled for 2 min, during which the centrifuge tube should not be shaken. 1 mL of an antibiotic-free LB liquid medium (10 g/L of tryptone, 5 g/L of yeast extract, and 5 g/L of sodium chloride) was added to each centrifuge tube, and a shake culture was conducted for 45 min in a 37° C. shaker at 180 rpm to make cells recovered. 100 μL of a resulting bacterial solution was pipetted and added to an LB solid medium including 100 μg/mL of Amp (ampicillin), and cells were gently spread evenly with a sterile pipette tip. The solid medium was air-dried on a clean bench, sealed with a plastic film, and invertedly cultured at 37° C. for 12 h to 16 h.

12 single colonies with pLB-TaGMS-A, pLB-TaGMS-B, or pLB-TaGMS-D transformed were picked with a toothpick, inoculated into 500 μL. of an LB liquid medium including 100 μg/ml of Amp, and cultured for 5 h under shaking at 37° C. and 200 rpm. All bacterial solutions produced were sent to the Sanger sequencing platform of Sangon Biotech (Shanghai) Co., Ltd. for sequencing. All single colonies were subjected to full-length sequencing coverage. Because the primers adopted to amplify the TaGMS-A B D genes are not much different from each other, the amplification of any gene in a subgenome is accompanied by the non-specific amplification of other two genes. Further, all sequences resulting from monoclonal sequencing were subjected to multi-sequence alignment, and target sequences could be divided into three types. Sequences of these three types could each be aligned with a corresponding gene in the reference genome of CS. The full-length sequences of TaGMS-A B D genes in the genome of Triticum aestivum L. Xichang 76-9 could be obtained according to the principle of highest homology similarity. The TaGMS-A B D gene sequences of Xichang 76-9 each were aligned with a coding sequence of a corresponding gene of CS to obtain TaGMS-A B D coding sequences of Xichang 76-9.

In the Sequence Listing:
SEQ ID NO: 1 indicates a nucleotide sequence of a TaGMS-A gene of Triticum aestivum L. “CS”;
SEQ ID NO: 2 indicates a coding sequence of a TaGMS-A gene of Triticum aestivum L. “CS”;
SEQ ID NO: 3 indicates a nucleotide sequence of a TaGMS-B gene of Triticum aestivum L. “CS”;
SEQ ID NO: 4 indicates a coding sequence of a TaGMS-B gene of Triticum aestivum L. “CS”;
SEQ ID NO: 5 indicates a nucleotide sequence of a TaGMS-D gene of Triticum aestivum L. “CS”;
SEQ ID NO: 6 indicates a coding sequence of a TaGMS-D gene of Triticum aestivum L. “CS”;
SEQ ID NO: 7 indicates a nucleotide sequence of a TaGMS-A gene of Triticum aestivum L. “Xichang 76-9”;
SEQ ID NO: 8 indicates a coding sequence of a TaGMS-A gene of Triticum aestivum L. “Xichang 76-9”;
SEQ ID NO: 9 indicates a nucleotide sequence of a TaGMS-B gene of Triticum aestivum L. “Xichang 76-9”;
SEQ ID NO: 10 indicates a coding sequence of a TaGMS-B gene of Triticum aestivum L. “Xichang 76-9”;
SEQ ID NO: 11 indicates a nucleotide sequence of a TaGMS-D gene of Triticum aestivum L. “Xichang 76-9”; and
SEQ ID NO: 12 indicates a coding sequence of a TaGMS-D gene of Triticum aestivum L. “Xichang 76-9”.

### Example 2. Analysis of Expression Patterns of TaGMS Genes in Triticum aestivum L.

With a RNAprep Pure plant total RNA extraction kit (TIANGEN (Beijing) Biotech Co., Ltd., Catalog No.: DP432), RNA was extracted from roots, stems, leaves, and anthers of Triticum aestivum L. Xichang 76-9 according to a method described in instructions of the kit. Three biological replicates were set for each tissue. With a Quant cDNA first-strand synthesis kit (TIANGEN (Beijing) Biotech Co., Ltd., Catalog No.: KR103), the extracted tissue RNA was reverse-transcribed according to a method described in instructions of the kit to synthesize cDNA.

### Reverse Transcription-Polymerase Chain Reaction (RT-PCR) Analysis

General primers for conserved regions of coding sequences of TaGMS-A BD genes of Triticum aestivum L. Xichang 76-9 were designed as follows:

TaGMS-CDs-F: 5′ TGGCTCACGAGCGGCAAC 3′ (SEQ ID NO: 19) (a target sequence in a TaGMS-A gene for this primer at the 15th bp was T, and one base mismatch generally did not affect the primer annealing); and

| | TaGMS-CD-R: |
|---|---|
| (SEQ ID NO: 20) | |
| | 5′ AATCTCCTGGCCAGTGCCCAT 3′ |

A fragment amplified with the primers TaGMS-CDs-F/R had a size of 325 bp.

With Triticum aestivum L. 18S RNA as an internal reference gene, primers were designed as follows:

| | a forward primer 18S-F: |
|---|---|
| (SEQ ID NO: 21) | |
| | 5′ CTGAGAAACGGCTACCACAT 3′; |
| | and |
| | a reverse primer 18S-R: |
| (SEQ ID NO: 22) | |
| | 5′ CCCAAGGTCCAACTACGAG 3′. |

A fragment amplified with the primers 18S-F/R had a size of 250 bp.

With the above primers, a TaGMS gene expression pattern was subjected to semi-quantitative analysis through PCR. A PCR system (30 μL) was as follows: 15 μL of 2×Taq Master Mix Dye (CoWin Biosciences, CW0682L), 0.8 μL of a forward primer (10 mM), 0.8 μL of a reverse primer (10 mM), 1 μL of cDNA, and 12.4 μL of ddH2O. A PCR program was as follows: pre-denaturation at 94° C. for 2 min; denaturation at 94° C. for 30 s, annealing at Tm−5° C. for 30 s, and extension at 72° C. for 10 s, with 34 cycles; and extension at 72° C. for 2 min. An annealing temperature for the TaGMS-CDs-F/R primer pair was 52° C., and an annealing temperature for the 18S-F/R primer pair was 50° C. After PCR was completed, 2% agarose gel electrophoresis was adopted to determine whether a target gene was expressed.

Quantitative Polymerase Chain Reaction (qPCR) Analysis

With Triticum aestivum L. 18sRNA as an internal reference, normalization was conducted, and expression levels of TaGMS-A/B/D) genes in anthers were analyzed by a Delta Delta CT relative quantitation method (2-ΔΔCT method). Specific primers for the TaGMS-A/B/D genes were designed separately as follows:

| | TaGMS-A-QPCR-F: |
|---|---|
| (SEQ ID NO: 23) | |
| | ATGATGAAACTAGTGGTGAT |
| | and |
| | TaGMS-A-QPCR-R: |
| (SEQ ID NO: 24) | |
| | TCCTTGCGGTGTTCTGACGA; |
| | TaGMS-B-QPCR-F: |
| (SEQ ID NO: 25) | |
| | CTTCAGCCTTGATCAGCAGAAACCA |
| | and |
| | TaGMS-B-QPCR-R: |
| (SEQ ID NO: 26) | |
| | AATGACTCGGTGACATACAAAACTA; |
| | and |
| | TaGMS-D-QPCR-F: |
| (SEQ ID NO: 27) | |
| | CAGCAATCCTACCGGTGACTTCAG |
| | and |
| | TaGMS-D-QPCR-R: |
| (SEQ ID NO: 28) | |
| | GACGGAAACTTTGAGAGCAGTAACA. |

PCR was conducted with a Super Real Pre Mix (SYBRGreen) kit (TIANGEN (Beijing) Biotech Co., Ltd., Catalog No.: FP205). A reaction system was as follows:

| | |
|---|---|
| 2 × SuperReal Pre Mix | 10 μl |
| Forward primer (10 μM) | 0.5 μl |
| Reverse primer (10 μM) | 0.5 μl |
| cDNA | 9 μl |

A reaction program was as follows: pre-denaturation at 94° C. for 30 s; and 94° C. for 5 s, 60° C. for 10 s, and 72° C. for 15 s, with 40 cycles. Each sample was assayed in triplicate with two technical replicates. Data was analyzed with the ROTOR6 software to obtain relative expression levels of TaGMS-A B D in anthers. A mean and standard deviation of three replicate experiments were calculated.

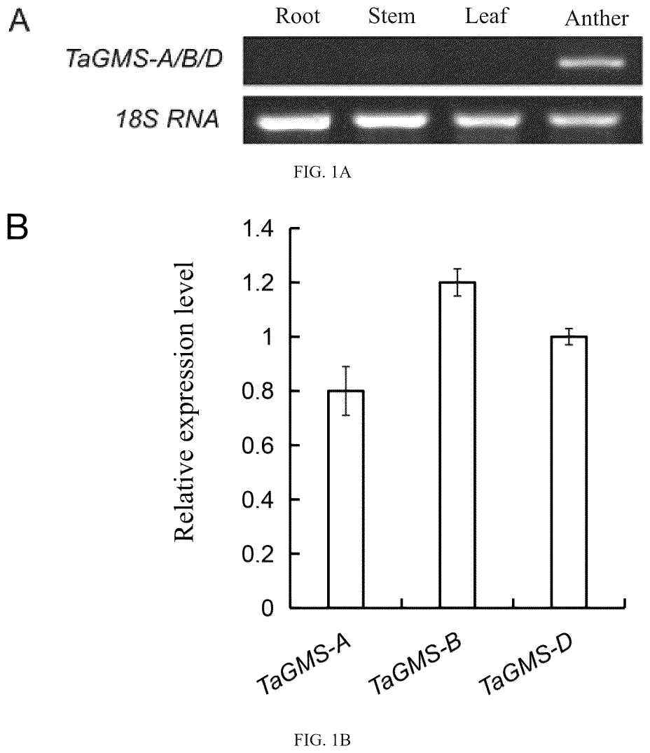

RT-PCR results showed that TaGMS-A BD genes were specifically expressed in anther tissues of Triticum aestivum L. Xichang 76-9, but were almost not expressed in roots, stems, and leaves (FIG. 1A). qPCR results showed that, in anthers of Triticum aestivum L. Xichang 76-9, a relative expression level of TaGMS-B was the highest, a relative expression level of TaGMS-D was in the middle, and a relative expression level of TaGMS-A was the lowest (FIG. 1B).

### Example 3. Acquisition of a Humidity-Sensitive Male-Sterile Material of Triticum aestivum L.

1. Mutagenesis of Triticum aestivum L. Xichang 76-9 with Sodium Azide (NaN3)

Xichang 76-9 (spring wheat) was selected as a donor plant. In March 2013, 50,000 full seeds of Xichang 76-9 were selected, soaked in distilled water at room temperature for 10 h, and then soaked in a phosphate-buffered saline (0.1 mM, pH=3) of 8 mM NaN3 for 3 h. During the soaking, the seeds were stirred to make the solution in full contact with the seeds. Soaked seeds were rinsed thoroughly under tap water for 2 h, drained, and sown in the Breeding Base of the Institute of Botany at the Chinese Academy of Sciences (Haidian, Beijing), which was denoted as an M0 generation. 3,000 M0 seeds were harvested in the year, which were denoted as an M1 generation. In April 2014, M1 lines were sown in the field, with 10 seeds for each line. After plants grew for one month A plant was randomly selected from each M1 line and labeled. Leaves were collected, and the corresponding plant DNA was extracted (a DNA extraction method was the same as above). The DNA was diluted to 50 ng/μL to 100 ng/μL for later use. After maturity, M2 seeds of this line were harvested. It was ensured that seeds of the M2 line corresponded to leaf DNA numbers of M1-generation plants. Single-mutant, double-mutant, and triple-mutant plants with different subgenomes of TaGMS-A B D genes were selected from an M2 mutant population established.

2. Screening of TaGMS-A/B/D Gene-Mutant Triticum aestivum L. Plants

TaGMS-A, TaGMS-B, and TaGMS-D-specific amplification primers and sequencing primers were designed as follows:

| Primer | | | Target |
|---|---|---|---|
| name | Primer sequence | Primer type | gene |
| 76-9 AF | CATATGTAGAGTTCT | Amplification | TaGMS- |
| | GATCTGTCAC | primers, | A |
| | (SEQ ID NO: 29) | product of | |
| | | 2,000 bp | |
| 76-9 AR | AGTTCAATATGTGAC | | |
| | CATTTTCTTGT | | |
| | SEQ ID NO: 30) | | |
| San-AF | CAGTTTGATGCTAGT | Sequencing | |
| | GAATA | primers | |
| | (SEQ ID NO: 31) | | |
| 76-9 BF | ATCTGACCACTTAAA | Amplification | TaGMS- |
| | TCCTCCTTC | primers, | B |
| | (SEQ ID NO: 32) | product of 691 | |
| | | bp | |
| 76-9 BR | GGAAGTGCAAGAATA | | |
| | TGAGAATAC | | |
| | (SEQ ID NO: 33) | | |
| San-BR | TCATCAGAGACCGCA | Sequencing | |
| | AAGGT | primers | |
| | (SEQ ID NO: 34) | | |
| 76-9 DF | GGTGGTATCTATCCA | Amplification | TaGMS- |
| | ACTGAATATTTGA | primers, | D |
| | (SEQ ID NO: 35) | product of | |
| | | 1,621 bp | |
| 76-9 DR | TTGAACAAGAAATTA | | |
| | ATCCAAATGTGAA | | |
| | (SEQ ID NO: 36) | | |
| San-DF | TCTGAACTGTATGCC | Sequencing | |
| | TGACGC | primers | |
| | (SEQ ID NO: 37) | | |

With the above amplification primers, the corresponding fragments of the target genes were amplified through PCR. High-fidelity enzymes used for PCR amplification all were ApexHF HS DNA Polymerase FS (Hunan Aikerui Biological Engineering Co., Ltd., Catalog No.: AG12202).

A PCR system was as follows: 15 μL of 2× Ape Mix, 1 μL of DNA, 0.6 μL of a forward primer (10 mM), 0.6 L of a reverse primer (10 mM), and 12.8 μL of ddH2O.

A PCR program was as follows: pre-denaturation at 94° C. for 30 s; denaturation at 98° C. for 10 s, annealing at 50° C. for 15 s, and extension at 72° C. for 2 min (an extension time for the 76-9 DF/DR primer pairs was 1 min), with 30 cycles; and final extension at 72° C. for 5 min.

With the above sequencing primers, an amplified fragment of each line was sequenced by the Sanger sequencing platform (Sangon Biotech (Shanghai) Co., Ltd.). The obtained sequences were aligned with TaGMS-A, TaGMS-B, and TaGMS-D gene sequences to select lines with mutation sites in exon regions.

Mutant sites of 3,000 Xichang 76-9 M2 plants were screened to obtain point-mutant plants in which there were nucleotide changes in coding regions of TaGMS-A/B/D genes. Names and mutant sites of the mutant plants were as follows:

S2034: A 3334th bp nucleotide of a TaGMS-A gene changed from G to A. A 1,595th nucleotide of a coding sequence of the gene changed from G to A. A 532nd codon changed from GGA to GAA. An amino acid encoded accordingly changed from glycine to glutamic acid (G/E).

S259: A 6th bp nucleotide of a TaGMS-B gene changed from G to A. A 6th nucleotide of a coding sequence of the gene changed from G to A. A 2nd codon changed from TGG to TGA. An amino acid encoded accordingly changed from tryptophan to a premature termination codon (W/*).

S924. A 1632nd bp nucleotide of a TaGMS-D gene changed from C to T. A 791st nucleotide of a coding sequence of the gene changed from C to T. A 264th codon changed from GCT to GTT An amino acid encoded accordingly changed from alanine to valine (A/V).

3. Pyramiding Crossing of Triticum aestivum L. TaGMS-A/B/D Mutants with Different Genotypes

With S2034 as a TaGMS-A gene donor mutant, S259 as a TaGMS-B gene donor mutant, and S924 as a TaGMS-D gene donor mutant, pyramiding crossing was conducted to obtain mutant materials with different TaGMS-A BD gene mutation combinations. A specific pyramiding crossing process was as follows:

In March 2016, about 20 seeds of each of S2034, S259, and S924 mutants were sown in the Molecular Breeding Base of the Institute of Botany at the Chinese Academy of Sciences. Leaf DNA of each mutant was extracted at a seedling stage. DNAs for all mutants S2034 were amplified with a 76-9 AF/AR primer pair, and then sequenced with primers San-AF to identify a homozygous mutant genotype of the TaGMS-A gene, which was named tagms-aaBBDD. DNAs for all mutants S259 were amplified with 76-9 BF/BR, and then sequenced with primers San-BR to identify a homozygous mutant genotype of the TaGMS-B gene, which was named tagms-AAbbDD. DNAs for all mutants S924 were amplified with a 76-9 DF/DR primer pair, and then sequenced with primers San-DF to identify a homozygous mutant genotype of the TaGMS-D gene, which was named tagms-AABBdd. Methods for genomic DNA extraction, PCR amplification, and genotype identification were the same as above. After each mutant entered a flowering period, tagms-aaBBDD and tagms-AAbbDD were subjected to pyramiding crossing, and at maturity, seeds were harvested to produce a heterozygous mutant of TaGMS-A and TaGMS-B, which was named tagms-AaBbDD. Moreover, tagms-AABBdd was bagged and self-pollinated, and seeds were harvested. In March 2017, about 20 seeds of each of the heterozygous tagms-AaBbDD and the homozygous tagms-AABBdd were sown in the Molecular Breeding Base of the Institute of Botany at the Chinese Academy of Sciences. After entering a flowering period, these two mutants were crossed, and at maturity, hybrid seeds F1 of this combination were harvested and screened to obtain a heterozygous mutant line with a combination of TaGMS-A, TaGMS-B, and TaGMS-D mutations. In March 2018, about 200 seeds F1 were sown in the Molecular Breeding Base of the Institute of Botany at the Chinese Academy of Sciences. After a line F1 entered a flowering period, genotyping was conducted to obtain a heterozygous mutant of tagms-AaBbDd. In March 2019, about 100 tagms-AaBbDd seeds were sown in the Molecular Breeding Base of the Institute of Botany at the Chinese Academy of Sciences. After entering a flowering period, plants were bagged and self-pollinated, and seeds were harvested at maturity. In March 2021, about 1,000 seeds resulting from the self-pollination of tagms-AaBbDd harvested in 2019 were sown in the Molecular Breeding Base of the Institute of Botany at the Chinese Academy of Sciences. At a seedling stage, genotyping was conducted to finally obtain all types of combined mutants of TaGMS-A, TaGMS-B, and TaGMS-D, which were single-mutant plants including tagms-aaBBDD, tagms-AAbbDD, and tagms-AABBdd, double-mutant plants including tagms-aabbDD, tagms-aaBBdd, and tagms-AAbbdd, and a triple-mutant plant tagms-aabbdd, and a WT material TaGMS-AABBDD with a relatively consistent genetic background. For each mutant line, mutant plants with consistent growth were selected, and bagged and self-pollinated in a flowering period, and seeds were harvested at maturity.

The tagms-aabbdd, tagms-aaBBDD, tagms-AAbbDD, and tagms-AABBdd had been deposited in the China General Microbiological Culture Collection Center (CGMCC), with deposit names of tagms1/tapts1-aabbdd, S2034, S259, and S924, respectively and accession numbers of CGMCC No. 45691, CGMCC No. 45692, CGMCC No. 45693, and CGMCC No. 45694, respectively.

4. Phenotypic Analysis of Different Triticum aestivum L. Mutants Based on Tagms

On Mar. 2, 2022, about 40 seeds of each of TaGMS-AABBDD (WT), tagms-aaBBDD, tagms-AAbbDD, tagms-AABBdd, tagms-aabbDD, tagms-aaBBdd, tagms-AAbbdd, and tagms-aabbdd were sown in a seedling tray, and the seedling tray was placed in the greenhouse of the Institute of Botany at the Chinese Academy of Sciences for seedling cultivation. After seedling emergence, seedlings were transplanted in the Molecular Breeding Base. For each material, 3 rows were sown, with 10 plants in each row, a plant spacing of 10 cm, and a row spacing of 25 cm. All materials underwent normal water and fertilizer management. After each material entered a flowering period, anthers of each line were collected, and fresh pollen was released on a glass slide. The glass slide was placed under a 10× lens of an optical microscope Olympus CX21 (Olympus, Japan) equipped with an image sensor CCD to observe morphological changes of pollen grains. The observed field of view was recorded in real time through photographing by the OPTPro software, and the photographing was conducted once every 5 s. At least three biological replicates were observed for each line. When pollen grains were observed, temperature and humidity changes were recorded in real time with a high-precision digital temperature and humidity meter (W8 Pro, Aicevoos). After the WT and all mutants were matured, seeds were harvested, and a number of seeds per ear was counted. 10 plants were selected from each line, and three ears were randomly selected from each plant to count a number of seeds per ear, with 30 ears in total. WT and all mutants were compared for the number of seeds per ear with one-way analysis of variance as a statistical method.

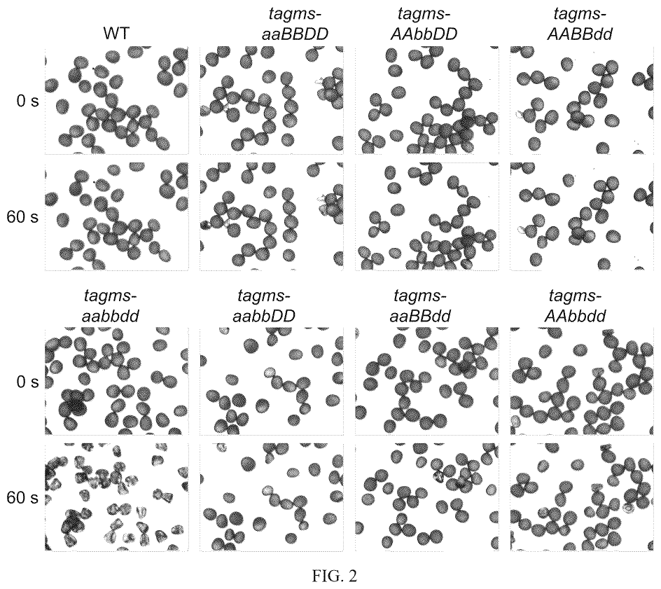

Results showed that, at 0 s after pollen grains were released from anthers (namely, immediately upon the detachment), pollen grains of all mutants and the WT had consistent morphologies without significant changes (FIG. 2). At 60 s after pollen grains were released from anthers, pollen grains of the WT, tagms-aaBBDD, tagms-AAbbDD, tagms-AABBdd, tagms-aabbDD, tagms-aaBBdd, and tagms-AAbbdd did not change significantly, while pollen grains of tagms-aabbdd all were shriveled (FIG. 2). In an environment for observing morphologies of pollen grains, a relative humidity was 40% to 55% and a temperature was 26° C. to 31° C.

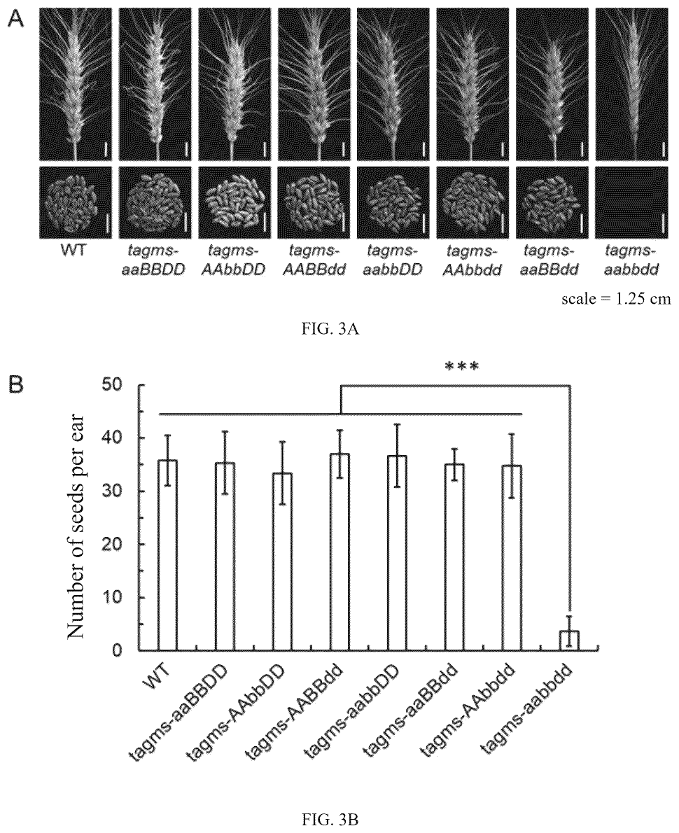

Results of natural ear seed setting analysis showed that there was no significant difference in ear seed setting between WT and tagms-aaBBDD, tagms-AAbbDD, tagms-AABBdd, tagms-aabbDD, tagms-aaBBdd, and tagms-AAbbdd, and an average number of seeds per ear was 33 to 37 (FIGS. 3A-3B). tagms-aabbdd had less than 3 seeds per ear on average and was almost sterile, which was very significantly different from other mutants (FIGS. 3A-3B).

5. Restoration of Fertility of the Triticum aestivum L. Mutant Tagms-Aabbdd

In a flowering period of Triticum aestivum L., 10 tagms-aabbdd mutant plants with basically the same growth were selected for a fertility restoration test. The mutant plant tagms-aabbdd was moisturized (humidity ≥90%) A method for the moisturizing was as follows: Before pollen shedding and after heading of the mutant plant tagms-aabbdd, the entire ear was covered with a transparent plastic bag or wrapped with a transparent plastic film (such as a plastic wrap), such that a humidity in an environment for the ear was no less than 90% until the pollen shedding was completed. Then the plastic bag or plastic film was removed. When seeds were mature, a number of seeds per ear of tagms-aabbdd after the moisturizing was counted, and 10 biological replicates were set. Moreover, Xichang 76-9 WT Triticum aestivum L. was moisturized in the same way.

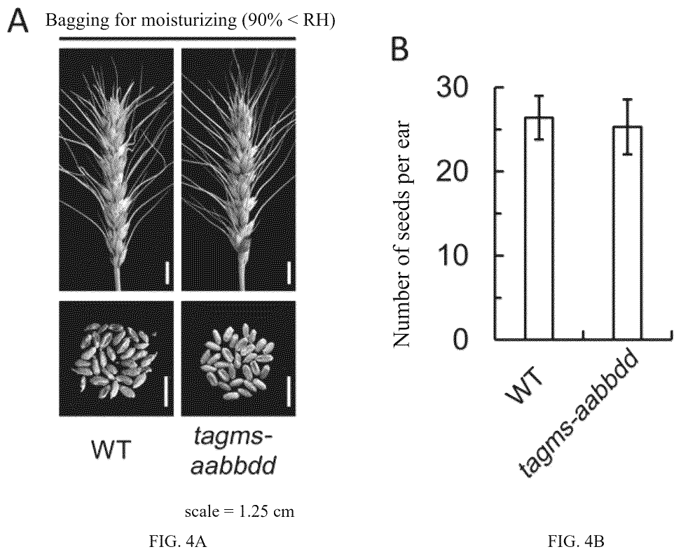

After ears of the mutant tagms-aabbdd were moisturized, numbers of seeds in 10 single ears were 25, 22, 30, 24, 21, 27, 23, 24, 31, and 26, respectively, and an average number of seeds per ear was 25.3±3.27 (FIGS. 4A-4B). After ears of Xichang 76-9 WT Triticum aestivum L. were moisturized, an average number of seeds per ear was 26.4±2.59 (FIGS. 4A-4B). The results showed that the seed setting of the mutant plant tagms-aabbdd could be restored by moisturizing ears before pollen shedding and after heading to maintain a humidity in an environment for the ears at 90% or more. Therefore, the tagms-aabbdd mutant is humidity-sensitive male-sterile Triticum aestivum L., which addresses the challenge in propagating sterile mutants and demonstrates a promising application prospect in the crossbreeding of Triticum destivum L.

What is claimed is:

1-12. (canceled)

13. A method for acquiring humidity-sensitive male-sterile Triticum aestivum L., comprising:
with Triticum aestivum L. as a donor plant, mutating a 1595th nucleotide of a coding sequence of a TaGMS-A gene from G to A, mutating a 6th nucleotide of a coding sequence of a TaGMS-B gene from G to A, and mutating a 791st nucleotide of a coding sequence of a TaGMS-D gene from C to T to produce a triple-mutant plant tagms-aabbdd as the humidity-sensitive male-sterile Triticum aestivum L.,
wherein the coding sequence of the TaGMS-A gene is shown in SEQ ID NO: 8;
the coding sequence of the TaGMS-B gene is shown in SEQ ID NO: 10; and
the coding sequence of the TaGMS-D gene is shown in SEQ ID NO: 12.

14. A method for acquiring humidity-sensitive male-sterile Triticum aestivum L., comprising:
subjecting first Triticum aestivum L. with an accession number of CGMCC No. 45692, second Triticum aestivum L. with an accession number of CGMCC No. 45693, and third Triticum aestivum L. with an accession number of CGMCC No. 45694 to a triallelic crossing and a self-pollinating to produce progeny plants; and
selecting a plant from the progeny plants to obtain a triple-mutant plant tagms-aabbdd as the humidity-sensitive male-sterile Triticum aestivum L., wherein in the plant, a 1595th nucleotide of a coding sequence of a TaGMS-A gene is A, a 6th nucleotide of a coding sequence of a TaGMS-B gene is A, and a 791st nucleotide of a coding sequence of a TaGMS-D gene is T,
wherein the coding sequence of the TaGMS-A gene is shown in SEQ ID NO: 8;
the coding sequence of the TaGMS-B gene is shown in SEQ ID NO: 10; and
the coding sequence of the TaGMS-D gene is shown in SEQ ID NO: 12.

15. A use of humidity-sensitive male-sterile Triticum aestivum L. with an accession number of CGMCC No. 45691 in a crossbreeding of Triticum aestivum L.

16. The use according to claim 15, wherein a method for improving a fertility of the humidity-sensitive male-sterile Triticum aestivum L. is as follows: during a flowering period of the humidity-sensitive male-sterile Triticum aestivum L., maintaining a humidity in an environment for an ear of the humidity-sensitive male-sterile Triticum aestivum L. at 90% or more.

17. The use according to claim 16, wherein a method for maintaining the humidity in the environment for the ear is as follows: wrapping the ear of the humidity-sensitive male-sterile Triticum aestivum L.

18. The use according to claim 17, wherein a method for the wrapping is as follows: before a pollen shedding and after a heading of the humidity-sensitive male-sterile Triticum aestivum L., covering the ear with a transparent plastic bag or wrapping the ear with a transparent plastic film until the pollen shedding is completed.

19. A method for improving a fertility of humidity-sensitive male-sterile Triticum aestivum L., comprising: during a flowering period of the humidity-sensitive male-sterile Triticum aestivum L., maintaining a humidity in an environment for an ear of the humidity-sensitive male-sterile Triticum aestivum L. at 90% or more, wherein the humidity-sensitive male-sterile Triticum aestivum L. is a triple-mutant plant tagms-aabbdd, wherein in the triple-mutant plant tagms-aabbdd, a 1595th nucleotide of a coding sequence of a TaGMS-A gene is mutated from G to A, a 6th nucleotide of a coding sequence of a TaGMS-B gene is mutated from G to A, and a 791st nucleotide of a coding sequence of a TaGMS-D gene is mutated from C to T;
the coding sequence of the TaGMS-A gene is shown in SEQ ID NO: 8;
the coding sequence of the TaGMS-B gene is shown in SEQ ID NO: 10; and
the coding sequence of the TaGMS-D gene is shown in SEQ ID NO: 12.

20. The method according to claim 19, wherein a method for maintaining the humidity in the environment for the ear is as follows: wrapping the ear of the humidity-sensitive male-sterile Triticum aestivum L.

21. The method according to claim 20, wherein a method for the wrapping is as follows: before a pollen shedding and after a heading of the humidity-sensitive male-sterile Triticum aestivum L., covering the ear with a transparent plastic bag or wrapping the ear with a transparent plastic film until the pollen shedding is completed.
